(19) United States
(12) Patent Application Publication
(10) Pub. No.: US 2025/0102378 A1
(43) Pub. Date: Mar. 27, 2025

(54) SURFACE/TACTILE SENSOR CONFIGURATIONS AND APPLICATIONS THEREOF

(71) Applicant: EZMEMS LTD., Caesarea (IL)

(72) Inventors: Nicola MOLINAZZI, Kfar Sava (IL); Tsvi SHMILOVICH, Pardes Hanna-Karkur (IL); Mark KANTOR, Ness Ziona (IL)

(21) Appl. No.: 18/833,861

(22) PCT Filed: Jan. 26, 2023
(86) PCT No.: PCT/IL2023/050093

§ 371 Date: Jul. 26, 2024

Related U.S. Application Data
(60) Provisional application No. 63/304,360, filed on Jan. 28, 2022.

(51) Int. Cl. G01L 1/22; G01L 25/00

(52) U.S. Cl. CPC G01L 1/2262; G01L 25/00

(57) ABSTRACT

A surface/tactile sensing device comprising an elongated interaction layer configured for interaction with environments external to the surface sensing device, one or more elongated gauging straps/patterns arranged along a length of the interaction layer to substantially traverse a deformable surface area of the surface sensing device, and at least one integration layer coating at least some portion of the surface sensing device substantially accommodating the deformable surface area interaction layer and configured to facilitate direct integration of the surface sensing device to an external surface area of an object.

### TECHNOLOGICAL FIELD

The present disclosure generally relates to sensor devices, particularly sensor devices usable for tactile sensory applications.

### BACKGROUND

This section intends to provide background information concerning the present application, which is not necessarily prior art.

Tactile sensors are designed for measurement of parameters/conditions effected by physical interaction with the surroundings environment e.g., utilizing piezoresistive, piezoelectric, capacitive and/or elastoresistive sensors. These types of sensors can be configured to mimic tactile sensory stimuli resulting from mechanical contact/pressure and/or temperature variations, which render them usable for robotic systems, touchscreen, security systems, and different various applications, such as but not limited to, recreational, sporting, military, wellness, gaming, diagnostic and medical.

There is an on growing market for pressure-responsive sensor devices that can be implemented in many different pressure sensing technologies. However, these devices are often limited because of not being flexible, and/or for not being suitable for use in curved/rounded surfaces and/or certain environments e.g., fluidic surroundings. Capacitive sensors, on the other hand, while being good candidates for flexible sensor implementations, they are typically easily infected by moisture/humidity e.g., when their surfaces are wet, which is critical for many applications. In addition, many of the solutions available nowadays don't offer a deterministic measurement e.g., they can just give an indication that there is a pressure or no, but they can't give a deterministic measurement i.e., a measured value.

Tactile sensors are typically manufactured as off-the-shelf products designed to be separately installed in consumer products requiring tactile sensory capabilities. However, the installation of separate tactile sensors in such products is not suitable for miniaturization and entails various disadvantages including, inter alia, low precision, inability to properly integrate with the components of the products, increased productions efforts and costs. Furthermore, these sensors are usually difficult and/or expensive to manufacture.

Various pressure/tactile sensor devices known from the patent literature are briefly described hereinbelow.

US Patent Publication No. 2003/199116 discloses an IC-integrated, flexible, shear-stress sensor skin made by providing a wafer with integrated circuits and sensor elements which are fabricated in the wafer, disposing a first polymer layer on the wafer and sensor elements to provide mechanical support for the sensor elements, defining a cavity below the sensor elements to provide thermal isolation, while the sensor element remains supported by the first polymer layer, and isolating the sensor elements into a plurality of islands defined in the wafer, so that the islands, with at least one sensor element on at least one of the islands, and the integrated circuits form the IC-integrated, flexible, shear-stress sensor skin.

US Patent Publication No. 2015/177080 discloses sensing devices including flexible and stretchable fabric-based pressure sensors associated with or incorporated in garments intended to be worn against a body surface (directly or indirectly), or associated with other types of flexible substrates, such as sheet-like materials, bandages, and other materials that contact the body (directly or indirectly), and may be provided as independently positionable sensor components. Systems and methods for storing, communicating, processing, analyzing and displaying data collected by sensor components for remote monitoring of conditions at body surfaces, or within the body, are also disclosed. Sensors and sensor systems provide substantially real-time feedback relating to current body conditions and may provide notifications or alerts to users, caretakers, and/or clinicians, enabling early intervention when conditions indicate intervention is appropriate.

US Patent Publication No. 2016/033343 discloses electronic skin pressure sensors and methods of using the same. Also disclosed an apparatus that includes an electronic skin pressure sensor and sensor circuitry. The electronic skin device is configured and arranged for differentiating between different mechanical stimuli including lateral stress and at least one additional mechanical stimuli. The sensor circuitry is configured and arranged to respond to the electronic skin pressure sensor by sensing a change in impedance due to the lateral stress.

### GENERAL DESCRIPTION

There is an ongoing demand for sensor devices that can be effectively and efficiently deployed over relatively large curved (or flat) surface areas, for measuring one or more parameters/conditions effected due to interaction with the surrounding environment. The currently available solutions are usually too bulky and/or expensive for realization, require substantial efforts for effective integration, and tend to compromise accuracy/sensitivity for the sake of miniaturization.

The present application discloses surface/tactile sensor implementations that are usable for tactile sensory, synthetic skin, medical/health monitoring/diagnostic and therapy, sport/muscle exercising, bio-feedback, temperature sensing, and suchlike applications. Other applications, such as, military, robotics, wellness, gaming, and/or security systems, are also conceivable. The term surface/tactile sensor is generally used herein to refer to flexible sensor devices that can measure one or more parameters and/or conditions affected by environmental surroundings over relatively wide curved/rounded and/or flat surface areas. The size of the surface area from which the one or more parameters and/or conditions can be measured from may vary according to the specific application requirements e.g., from few square millimeters, or centimeters, and up to few square meters.

One aspect of the present application is directed to a surface/tactile sensing device comprising an interaction layer configured for interaction with environments external to the device, one or more gauging straps/patterns arranged on or in the interaction layer to substantially cover a surface area thereof, and at least one integration layer coating at least some portion of the interaction layer and configured to facilitate attachment thereof to an external surface area of an object.

The one or more gauging straps/patterns comprises in some embodiments at least one sensing strap/pattern and at least one reference strap/pattern used as a reference for measured signals/data from the at least one sensing strap/pattern. The surface/tactile sensing device can comprise at least two of the sensing straps/patterns and at least two of the reference straps/patterns. Optionally, the at least one sensing strap/pattern and the at least one reference strap/pattern are configured to implement a bridge measurement circuitry.

In some embodiments the surface/tactile sensing device comprises an elongated interaction layer configured for interaction with environments external to the surface sensing device, one or more elongated gauging patterns arranged along a length of the elongate interaction layer to substantially traverse a deformable surface area of the surface sensing device, and at least one integration layer coating at least some portion of the surface sensing device substantially accommodating the deformable surface area and configured to facilitate direct integration of said surface sensing device to an external surface area of an object. The surface/tactile sensing device can be configured for attachment over a flat surface area of the object, or over a rounded surface area of the object such that its one or more elongated gauging patterns assume an annular geometry traversing a substantial circumference of the rounded surface area.

Optionally, but in some embodiments preferably, the one or more elongated gauging patterns comprise at least one elongated sensing pattern and at least one elongated reference pattern arranged along a length of the interaction layer substantially parallel to the at least one sensing pattern, and configured to provide a reference for measurement signals/data from the at least elongated one sensing strap/pattern. The surface/tactile sensing device can be configured to locate the elongated sensing patterns over an elongated cavity of the object defining a deformable sensing region of the device. In possible applications the at least one elongated reference pattern is arranged on a surface area of the elongated interaction layer configured for reduced or non-observable deformations. The surface/tactile sensing device comprises in some embodiments at least two of the elongated sensing patterns and at least two of the elongated reference patterns.

The at least one elongated sensing pattern and the at least one elongated reference pattern can be configured for bridge circuitry measurements. The voltage dividing branches of the bridge circuitry comprises in some embodiments at least one elongated sensing pattern and at least one elongated reference pattern. For example, one of the voltage dividing branches can be arranged to present voltage over one of the elongated sensing patterns and another one of the voltage dividing branches can be arranged to present voltage over one of the elongated reference patterns.

The surface/tactile sensing device comprises in some embodiments an electrical connection region at one end of the elongated interaction layer. The electrical connection region can be configured to electrically connect to at least one of the one or more elongated gauging patterns. The surface/tactile sensing device can include elongated electrically conducting lines arranged along a length of the elongated interaction layer and configured to electrically connect between electrically conducting lines of the electrical connection region and extremities of the elongated gauging patterns at another end of the elongated interaction layer.

The surface/tactile sensing device can comprise one or more vias configured to electrically connect between the elongated gauging patterns arranged on one side of the elongated interaction layer and at least one of the electrically conducting lines of the electrical connection region and the elongated electrically conducting lines arranged on another side of the electrical connection region. Electrically connecting patterns can be used in the surface/tactile sensing device to connect the one or more vias to the elongated gauging patterns. Optionally, the electrically connecting patterns are at least partially arranged on extremities of the elongated gauging patterns. Alternatively, the elongated gauging patterns are at least partially arranged on extremities of the electrically connecting patterns.

The surface/tactile sensing device can be configured for interfacing to a control unit configured to at least one of a calibration procedure for calibrating the bridge circuitry and communication of measurement data/signals from the bridge circuitry. The surface/tactile sensing device comprises in some embodiments a connectivity module configured to communicate the measurement data/signals to the control unit wirelessly or over data/signal communication lines.

In possible embodiments the surface/tactile sensing device comprises one or more tuneable resistive elements electrically coupled to at least one voltage dividing branch of the bridge circuitry. The calibration procedure may comprise adjusting electrical resistance of the one or more tuneable resistive elements by the control unit until the bridge circuitry is substantially balanced. In some applications the control unit is configured with a surface area associated with the one or more elongated gauging patterns to distinguish between application of pressure/force over one or more surfaces on the device and application of pressure/force over a discrete point on the device.

The surface/tactile sensing device comprises in some embodiments an adhesive layer connecting the at least one integration layer to the elongated interaction layer, and/or at least one cover layer configured to cover the elongated gauging patterns and communicate external pressure or forces applied thereover to the interaction layer. The at least one cover layer can be configured to improve sensing sensitivity of the device. Optionally, but in some embodiments preferably, the surface/tactile sensing device comprises an isolation layer configured between the at least one cover layer and the interaction layer.

One or more of the elongated gauging patterns are used in some embodiments for measuring changes of one or more electrical and/or electrochemical properties/conditions of a medium or environment external to said device. Optionally, but in some embodiments preferably, the surface sensing device comprises an antenna and related circuitries for one or both energy harvesting and communicating measurement data/signals with an external device.

Another aspect of the present application is directed to a circumferential sensing device comprising at least one surface/tactile sensing device according to any of the embodiments disclosed herein attached at least partially to a curved/circular portion of the object. The circumferential sensing device can be configured to provide for deformations of at least a portion of the surface/tactile sensing device.

The surface/tactile sensing device comprises in some embodiments a soft/flexible layer covering at least a portion thereof configured for interaction with external environment thereof.

Yet another aspect of the present application is directed to a surface/tactile sensing device comprising an elongated rigid core, one or more soft/flexible cover layers applied over at least some portion of said elongated rigid core, and a plurality of sensor devices embedded in or on said one or more soft/flexible cover layers.

The surface/tactile sensing device of any of the embodiments described herein can be configured for attachment over a cavity or fluid passage for allowing deformations of at least some portion of the sensing device in response to applied force/pressure.

The surface/tactile sensing device may comprise at least one electrically conducting layer configured to provide electrical connectivity between and to the one or more sensing and/or reference straps/patterns. Optionally, at least a portion of the at least one electrically conducting layer at least partially overlaps at least a portion of one of the sensing and/or reference straps/patterns.

A yet other aspect of the present application is directed to a calibration unit comprising at least one controllably adjustable electrical resistance element electrically connectable to a sensing device, a measurement unit configured to acquire measurement data/signals from the sensing device under predefined operational condition thereof, and a control unit configured to adjust electrical resistance of the controllably adjustable electrical resistance element in accordance with the measurement data/signals to correspondingly set the measurement data/signals to reside within a predefined range associated with the predefined operational condition of said sensing device. The calibration device can be used for calibration of the sensing device according to any of the embodiments disclosed herein, or for calibration of a bridge circuitry comprising these sensing devices.

The surface sensing device may comprise multiple layers of said surface sensing device arranged one on top of the other. In possible embodiments the surface sensing device comprises one or electrodes configured to contact a medium and measure electrical conductivity thereof. Additionally, or alternatively, the surface sensing device comprises one or optical sensing arrangement configured to measure light transmittance and/or reflectance of a medium.

Yet in another aspect there is provided a circumferential sensing device comprising at least one surface/tactile sensing device configured according to any of the embodiments disclosed herein attached at least partially to a curved or flat portion of the object. The circumferential sensing device can be configured for at least one of exercising and/or diagnosing annular muscles of a treated/exercised subject. The circumferential sensing device comprises in some embodiments an elongated rigid core, one or more soft/flexible cover layers applied over at least some portion of the elongated rigid core structure, and a plurality of the surface sensing devices embedded in or on the one or more soft/flexible cover layers and along the elongated rigid core. The circumferential sensing device can have one or both of the following configurations: (i) the elongated core is made substantially stiff and/or rigid; and (ii) the one or more soft/flexible cover layers comprising or carrying one or more reinforcing elements each configured to surround and support at least some portion of one of the surface sensing devices. Optionally, the circumferential sensing device comprises an actuatable portion having the at least one surface sensing device annularly arranged over a cylindrical portion thereof, and a ball portion movable coupled to the actuatable portion.

In yet another aspect there is provided a casting apparatus for applying a cover layer over the circumferential sensing device of any of the embodiments disclosed herein. The casting apparatus comprises connectable front and rear portions having cavities configured to hold components of the circumferential surface sensing device substantially centered thereinside, and an injection port provide in at least one of said front and rear portions and configured for injection of a curable casting material to the cavities.

In yet another aspect there is provided a method of preparing a sensing device. The method comprising applying at least one integration layer over at least some portion of an elongated interaction layer for coating at least some portion thereof substantially accommodating a deformable surface area of the elongated interaction layer, the elongated interaction layer configured for interaction with environments external to the sensing device and the at least one integration layer configured to facilitate direct integration of the sensing device to an external surface area of an object, and arranging one or more elongated gauging patterns along a length of said elongated interaction layer to substantially traverse its deformable surface area.

The arranging of the one or more elongated gauging patterns comprises in some embodiments forming at least one elongated sensing pattern and at least one elongated reference pattern along a length of the interaction layer substantially parallel to the at least one sensing pattern. The method can comprise forming the elongated sensing patterns over a region of the elongated interaction layer configured to cover an elongated cavity of an object defining a deformable sensing region of the device. In possible applications the method comprises arranging the at least one elongated reference pattern on a surface area of the elongated interaction layer configured for reduced or non-observable deformations. The method can comprise arranging at least two of the elongated sensing patterns, and at least two of the elongated reference patterns, on the elongated interaction layer.

The method comprises in some embodiments configuring the at least one elongated sensing pattern and the at least one elongated reference pattern for bridge circuitry measurements. The method optionally comprising arranging the bridge circuitry such that voltage dividing branches thereof comprise at least one elongated sensing pattern and at least one elongated reference pattern. For example, the bridge circuitry can be arranged such that one of the voltage dividing branches presents voltage over one of the elongated sensing patterns and another one of the voltage dividing branches presents voltage over one of the elongated reference patterns.

The method comprising in some embodiments forming an electrical connection region at one end of the elongated interaction layer, and configuring the electrical connection region to electrically connect to at least one of the one or more elongated gauging patterns. The method can comprise arranging elongated electrically conducting lines along a length of the elongated interaction layer and electrically connecting between electrically conducting lines of the electrical connection region and extremities of the elongated gauging patterns at another end of the elongated interaction layer.

In possible embodiments the method comprising forming one or more vias in the elongated interaction layer and electrically connecting through the one or more vias between the elongated gauging patterns arranged on one side of the elongated interaction layer and at least one of the electrically conducting lines of the electrical connection region and the elongated electrically conducting lines arranged on another side of the electrical connection region. The method can comprise forming electrically connecting patterns on the elongated interaction layer and at least partially on extremities of the elongated gauging patterns for electrically connecting the one or more vias to the elongated gauging patterns. The method may comprise arranging the elongated gauging patterns at least partially on extremities of the electrically connecting patterns.

The method comprising in some embodiments connecting the at least one integration layer to the elongated interaction layer by an adhesive layer. The can comprise applying at least one cover layer over the elongated gauging patterns and configuring at least one cover layer to communicate external pressure or forces applied thereover to the interaction layer. The method optionally comprising applying an isolation layer between the at least one cover layer and the interaction layer.

In yet another aspect there is provided a method of measuring one or more properties or conditions external to or acting on an object or medium. The method comprising attaching over a flat or rounded surface area of the object or medium one or more surface/tactile sensing devices according to any of the embodiments disclosed herein, and acquiring measurement data from the elongated gauging patterns and/or electrodes and/or optical sensing arrangements of the one or more surface sensing devices responsive to pressure or force applied thereover. The object can comprise an elongated core, and the method can comprise applying one or more soft/flexible cover layers over at least some portion of the elongated core structure, and embedding the one or more surface sensing devices in or on the one or more soft/flexible cover layers and along the elongated rigid core. The method comprising in some embodiments forming one or more reinforcing elements in the one or more soft/flexible cover layers, and configuring each of the reinforcing elements to surround and support at least some portion of one of the surface sensing devices.

The method can comprise interfacing a control unit to at least one of the surface sensing devices and using the control unit for calibrating the at least one surface sensing device and/or communicating measurement data/signals therefrom. The method comprising in some embodiments configuring the control unit with a surface area associated with the one or more elongated gauging patterns to distinguish between application of pressure/force over one or more surfaces on said device and application of pressure/force over a discrete point on said device. The method can comprise interfacing a respective connectivity module to at least some of the surface sensing devices and communicating the measurement data/signals to the control unit wirelessly or over data/signal communication lines by the respective connectivity module.

In some embodiments the method comprising configuring the control unit to adjust one or more tuneable resistive elements electrically coupled to at least one voltage dividing branch of a bridge circuitry implemented in at least one of the surface sensing devices until the bridge circuitry is substantially balanced. The method can comprise placing the object in a cavity or passage in a body of a treated subject, and using the measurement data for diagnosing said cavity or passage and/or for exercising annular muscles associated with said cavity or passage. The method optionally comprising centering the object with the surface sensing devices thereby carried in one or more cavities defined inside a casting device and injecting a curable casting material into the cavities via an injection port provide of the casting device.

In possible applications the method comprises measuring one or more properties or conditions of the medium at least partially enclosed by the one or more surface sensing devices.

### BRIEF DESCRIPTION OF THE DRAWINGS

In order to understand the invention and to see how it may be carried out in practice, embodiments will now be described, by way of non-limiting example only, with reference to the accompanying drawings. Features shown in the drawings are meant to be illustrative of only some embodiments of the invention, unless otherwise implicitly indicated. In the drawings like reference numerals are used to indicate corresponding parts, and in which:

FIGS. 1A to 1D schematically illustrate a surface/tactile sensor device and various possible applications thereof according to some possible embodiments, wherein FIG. 1A schematically illustrates possible structure of the surface/tactile sensor device, FIG. 1B shows a sectional view of the surface/tactile sensor device coupled to an object, FIG. 1C shows a bridge circuitry implemented utilizing the surface/tactile sensor configuration of FIG. 1B, and FIG. 1D exemplifies a curved/rounded configuration of the surface/tactile sensor device;

FIGS. 2A and 2B schematically illustrate multilayered surface/tactile sensor implementations according to some possible embodiments, wherein FIG. 2A shows a multilayered surface/tactile sensor structure having a cavity configured for sensing external pressures/forces and FIG. 2B shows a multilayered surface/tactile sensor structure having a cavity connected to a fluid channel for sensing differential of internal and external pressures/forces;

FIG. 3 schematically illustrates a sensing catheter device having a plurality of surface/tactile sensing elements according to some possible embodiments;

FIGS. 4A to 4D schematically illustrate structure of a surface/tactile sensor design according to some possible embodiments, wherein FIG. 4A shows a top view of the surface/tactile sensor device, FIG. 4B shows a bottom view of the surface/tactile sensor device, FIG. 4C shows an extremity of the top view of the surface/tactile sensor device, and FIG. 4D demonstrates a possible attachment scheme of the bottom side of the surface/tactile sensor device to a curved/rounded or flat surface;

FIGS. 5A and 5B schematically illustrate structure of a multilayered surface/tactile sensor design according to other possible embodiments, wherein FIG. 5A shows a sectional view of the surface/tactile sensor device and FIG. 5B shows various layers of the surface/tactile sensor device;

FIGS. 6A and 6B show simulation results obtained for the surface/tactile sensor configurations of FIGS. 4 and 5 when external pressure is applied over a hollow/cylindrical circular object carrying the surface/tactile sensor, wherein FIG. 6A shows the surface/tactile sensor before application of the pressure thereover, and FIG. 6B shows the surface/tactile sensor after application of the pressure thereover;

FIGS. 7A to 7C schematically illustrate circuitries for interfacing and calibrating the surface/tactile sensor device according to some possible embodiments, wherein FIG. 7A exemplifies connectivity to the surface/tactile sensor device, FIG. 7B shows a possible measurement circuitry, and FIG. 7C is a flowchart demonstrating possible offset compensation, calibration and measurement schemes usable for the surface/tactile sensor devices disclosed herein;

FIGS. 8A to 8C schematically illustrate possible applications utilizing the surface/tactile

sensor device according to possible embodiments, wherein FIG. 8A shows a possible communication and power module of the surface/tactile sensor device, FIG. 8B demonstrates data communication application, and FIG. 8C demonstrates application of multiple surface/tactile sensing devices and power/connectivity modules configured for sensing over continuous wide areas;

FIG. 9 schematically illustrates a surface/tactile sensor design according to yet other possible embodiments;

FIG. 10A to 10F are sectional views of possible surface/tactile sensor layered structures, wherein FIG. 10A shows an isolation/integration layer/substrate with two electrically conducting layers applied over two opposite side surfaces thereof, FIG. 10B shows an isolation/integration layer/substrate with two electrically conducting layers applied over two opposite sides thereof and two protective/integration layers, each applied over a respective one of the electrically conducting layers, FIG. 10C shows the layered structure of FIG. 10B with sensing layers sandwiched between the isolation/integration layer/substrate and the conductive layers, FIG. 10D shows the layered structure of FIG. 10C with multiple conductive and isolation/integration layers, FIG. 10E exemplifies a possible surface/tactile sensor construction scheme, and FIG. 10F exemplifies a possible modification of the surface/tactile sensor device of FIG. 10C;

FIG. 11 schematically illustrates a possible application of sensor devices according to possible embodiments;

FIGS. 12A to 12C schematically illustrate another surface/tactile sensor configuration according to some possible embodiments, wherein FIG. 12A shows a sectional view of a possible surface/tactile sensor configuration, and FIGS. 12B and 12C respectively show sectional side and bottom views of a modified surface/tactile sensor configuration;

FIGS. 13A to 13H schematically illustrate a practice device utilizing a surface/tactile sensor, and a possible manufacturing technique thereof, according to possible embodiments, wherein FIGS. 13A to 13E show structure and components of actuatable portion of the practice device and FIGS. 13F to 13H shows the final device and its deformable enclosure;

FIGS. 14A to 14F schematically illustrate casting process and equipment for applying the deformable enclosure over the components of the practice device shown in FIGS. 13A to 13H, wherein FIG. 14A shows a mold casting apparatus usable for encasing the practice device in a mold, FIGS. 14B and 14C show sectional views of the mold casting apparatus, FIG. 14D shows cable holder apparatus of the mold casting apparatus, FIG. 14E demonstrates use of spacers for centering portions of the practice device in their respective mold casting cavities, and FIG. 14F is a flowchart illustrating steps of the mold casting process; and

FIGS. 15A and 15B schematically illustrate additional sensor configurations.

### DETAILED DESCRIPTION OF EMBODIMENTS

The present application provides techniques for sensing metrics (parameters and/or conditions) effected due to interaction of an object with its surrounding environment. For this purpose, the inventors hereof developed new flexible sensor designs configured to be easily integrated into appliances/devices, for readily and effectively collecting measurement data/signals from relatively large external surface areas of the appliances/devices that interacts with the external environment. The sensor designs disclosed herein are thus sometimes referred to herein as surface sensors, or tactile sensors, or generally, as sensor devices.

In a broad aspect, the surface/tactile sensors disclosed herein are made in a form of flexible sensing sheets having one or more gauging straps and/or patterns arranged therein in one or more orientations for sensing one or more parameters and/or conditions effected due to interaction of the sheets with the surrounding environment. The gauging straps/patterns can be arranged in flexible sheets to form straight, curved, wavy, crossing, patterns, according to specific application requirements. The flexible sheets with their gauging straps/patterns can be quickly and easily attached over curved/rounded or flat surfaces of objects for sensing their interaction with the surrounding environment. In some embodiments the flexible sensing sheets are multilayered structures incorporating their gauging straps/patterns in a protective and operative manner for improved flexibility and gauging sensitivity.

One or more specific and/or alternative embodiments of the present disclosure will be described below with reference to the drawings, which are to be considered in all aspects as illustrative only and not restrictive in any manner. It shall be apparent to one skilled in the art that these embodiments may be practiced without such specific details. In an effort to provide a concise description of these embodiments, not all features or details of an actual implementation are described at length in the specification. Elements illustrated in the drawings are not necessarily to scale, or in correct proportional relationships, which are not critical. Emphasis instead being placed upon clearly illustrating the principles of the invention such that persons skilled in the art will be able to make and use the sensor devises and realize their implementations/applications, once they understand the principles of the subject matter disclosed herein. This invention may be provided in other specific forms and embodiments without departing from the essential characteristics described herein.

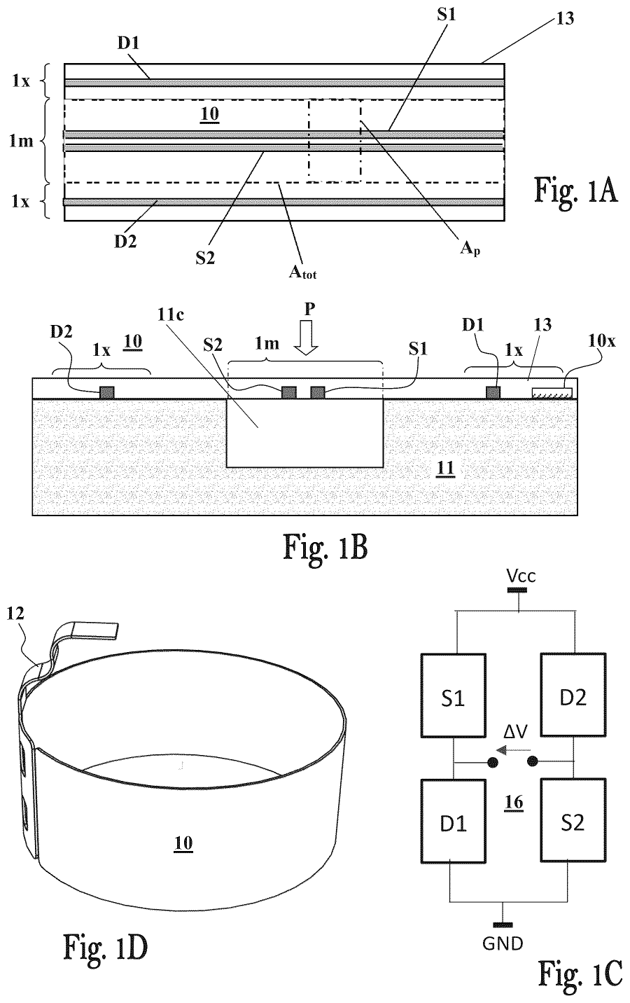

FIG. 1A schematically illustrates a surface/tactile sensor 10 according to some possible embodiments. The sensor device 10 is made of a flexible/deformable sheet 13 and one or more gauging straps/patterns D1, S1, D2, S2, . . . arranged therein in one or more orientations. In this specific and non-limiting example the gauging straps/patterns D1, S1, D2, S2, . . . are straight straps arranged in parallel along the length of the sheet 13, but other orientations (e.g., curved, waving, crossing) of the gauging straps D1, S1, D2, S2, . . . can be similarly and easily devised.

In some embodiments the sensing sheet 13 is made from flexible/deformable (e.g., polymeric) material(s), such as, but not limited to, polyimide (e.g., Kapton), PEEK, fiber reinforced composite materials, PET, PC, Silicone, Rubber, or from other suitable polymeric material, or from a multilayered structure comprising such materials. The thickness of the sensing sheet 13 can generally be in the range of 0.005 to 5 millimeters, and it can be configured for coupling/attaching to curved surface areas of objects. Same/similar configurations of the surface/tactile sensor 10 can be implemented on/in non-flexible sheets 13 e.g., made of stiff, or elastic, or resilient materials, which are more suitable for coupling to substantially flat surfaces.

The sensing sheet 13 and its one or more gauging straps/patterns D1, S1, D2, S2, . . . can be manufactured using any of the techniques disclosed herein or in International Patent Application Nos. WO 2015/114635, WO 2018/025264, WO 2019/171376, WO 2018/235087, WO 2020/095309, WO 2020/129069, of the same Applicant hereof, the disclosures of which is incorporated herein by reference.

The gauging straps/patterns D1, S1, D2, S2, . . . are configured in some embodiments to change one or more electrical properties thereof (e.g., resistance, inductance, capacitance) in response to physical interaction with the environment (e.g., responsive to temperature changes, applied pressure/force, etc.). In some embodiments the gauging straps/patterns D1, S1, D2, S2, . . . are configured to form strain gauges e.g., made of polymeric based material (e.g., carbon or graphene bases), metal (e.g., NiCr, Constantan, etc.), having thickness of about 0.00001 to 1 millimeters, and configured to deform and adapt the shape/curvature of the flexible sensing sheet 13. Though the gauging straps/patterns D1, S1, D2, S2, . . . shown in FIG. 1A are exemplified as straight and substantially parallel straps, they can be similarly have wavy, curly or curved configuration.

FIG. 1B shows a sectional view of the sensor device 10 when coupled to an object (base) element 11. In this non-limiting example the gauging straps/patterns S1 and S2 are placed over a cavity (or channel) 11c formed in the object 11 e.g., filled with a fluid/air (to form the measuring region 1m), such that they are readily effected by external pressures P exerted over the surface/tactile sensor device 10, while the gauging straps/patterns D1 and D2 are placed over substantially rigid portions of the object 11 (fixed regions 1x), and thus the effect of the external pressures P over the gauging straps/patterns D1 and D2 is negligibly small/non-observable or zero.

In this specific and non-limiting example, the gauging straps/patterns S1 and S2 located over the cavity (or channel) 11c are used as sensing straps, and the straps/patterns D1 and D2 located over the rigid portions (1x) of the object/base 11 (areas not affected by deflections of the sensing sheet e.g., the areas used to attach/adhere the sensing sheet 13 to the object/base 11) can be used as reference straps/patterns. In possible embodiments the reference straps/patterns D1 and D2 are implemented by the same material/configuration of the sensing straps/patterns S1 and S2, generally having a width in a range of 0.0005 to 2 millimeters, optionally about 0.10 millimeters, and a thickness in a range of 0.001 to 500 μm (micrometers), optionally about 20 μm. The sensing straps/patterns S1 and S2 can be implemented by piezoresistive straps/patterns (e.g., made of polymeric based material, such as carbon or graphene based, or metallic materials, such as NiCr, Constantan, etc.) having a width in a range of 0.0005 to 2 millimeters, optionally about 0.15 millimeters, and a thickness in a range of 0.001 to 500 μm, optionally about 20 μm.

For example, FIG. 1C shows a bridge circuitry 16 implemented by the sensor device configuration of FIG. 1B, wherein each voltage dividing branch of the bridge circuitry 16 comprises one sensing strap/pattern, S1 or S2, electrically connected to one of the reference straps/patterns, D1 or D2, respectively. The voltage AV measured between the two voltage dividing branches is indicative of the pressure P over the sensing straps/patterns S1 and S2. In order to maximize the sensitivity of the bridge circuitry 16, one voltage dividing branch is arranged to present the voltage over its reference strap/pattern (e.g., D1), and the other voltage dividing branch is arranged to present the voltage over its sensing strap/pattern (e.g., S2). Though FIG. 1C exemplifies a half bridge configuration, in possible embodiments the sensor design can be configured to implement a full bridge (e.g., utilizing compression and extension gauges).

In some embodiments the sensing straps/patterns S1 and S2 are configured to present a certain electrical resistance Rₜₒₜ, representing a relaxed state in which interaction with the external environment is minimal, or not effecting the sensor device 10. When a pressure P is applied over a surface area A of the surface/tactile sensor 10, the applied pressure changes are proportional to the resistance changes effected in the sensing straps/patterns—P˜ΔRₜₒₜ/Rₜₒₜ. Thus, if a specific pressure level (P1) is applied over a smaller surface area of the sensor device e.g., Ap=(1/10)·Aₜₒₜ, the exerted pressure causes electrical resistance changes only in the effected area i.e., ΔRₚ/Rₜₒₜ where Aₜₒₜ is the surface area portion of the sensor device 10 covering the cavity/channel 11c (also referred to herein as the sensing surface or area), and Rₚ=(1/10)·Rₜₒₜ, such that P1˜ΔRₚ/Rₜₒₜ. If the same level of pressure is applied over the entire sensing surface Aₜₒₜ, then the total resistance change would be higher and so the output of the bridge circuit 16.

If the pressure P1 is exerted over the entire surface of the sensing area Aₜₒₜ of the surface/tactile sensor 10, the measured resistance change is maximized. On the other hand, if the surface/tactile sensor device 10 is implemented only in a restricted area Ap, and pressure is exerted outside this area, it will not be sensed by the sensor device. This reflects the difference between surface sensing (sometimes referred to herein as circumferential sensing) to “point pressure measurement”.

FIG. 1D exemplifies a curved/rounded configuration of the sensor device 10 having an electrical connection band 12 laterally extending from the sensor device 10 and configured to provide electrical connectivity to at least one of the gauging straps/patterns D1, S1, D2, S2, . . . .

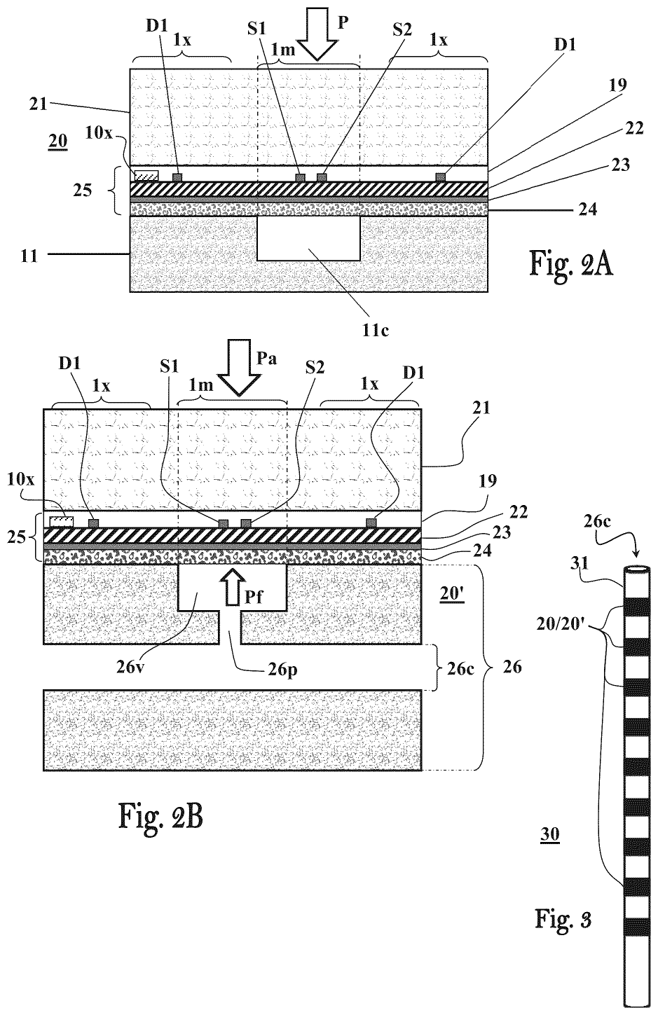

FIG. 2A shows a sectional view of a surface/tactile sensor device 20 having a multilayered sensing structure 25 attached to a base/cap component 11 having a cavity/fluid channel 11c, configured for sensing external pressures/forces P. The sensor device 20 comprises a deformable external layer 21 (also referred to herein as cover layer e.g., made of flexible material(s) e.g., a type of Rubber, liquid silicon, any other suitable silicon materials, or suchlike) attached to one side of the multilayered structure 25, and a substantially rigid base/object (e.g., made from a type of plastic material) 11 having the cavity/fluid channel 11c attached to the other side of the multilayered structure 25, such that the cavity/fluid channel 11c is thereby sealably closed.

The deformable external layer 21 can be manufactured from any suitable material with flexible/deformable/elastic properties, optionally from a type of soft material (e.g., rubber, liquid silicon, or any other suitable silicon materials), and its thickness can generally be in the range of 0.1 to 50 millimeters. The multilayered structure 25 comprises in some embodiments an isolation/integration layer 19 (e.g., made from dielectric isolator material, such as thermoplastic or thermoset, and having a thickness generally in the range of 0.0001 to 3 millimeters) substantially covering the gauging straps/patterns D1, S1, D2, S2, . . . , a substrate layer 22 (also referred to herein as interaction layer e.g., made from a polyimide such as Kapton, PEEK, fiber reinforced composite materials, PET, PC, Silicone, Rubber, or from other suitable polymeric materials, and having a thickness generally in the range of 0.0005 to 5 millimeters) on/in which the gauging straps/patterns D1, S1, D2, S2, . . . , are formed/implemented, and an (optional) integration layer 24 (e.g., made from dielectric isolator material, thermoplastic or thermoset and having a thickness generally in the range of 0.0001 to 5 millimeters) adapted to connect between the multilayered structure 25 and the base element/object 11. An adhesive layer 23 (e.g., an epoxy or acrylic based adhesive, or any other suitable adhesive, having a thickness generally in the range of 0.0001 to 5 millimeters) is used in some embodiments to connect between the substrate layer 22 and the integration layer 24.

Optionally, but in some embodiments preferably, the isolation/integration layer 19 is a multilayered structure comprising various different layers having different properties and/or made of different materials. For example, the isolation/integration layer 19 can have a main layer configured for direct integration with the deformable external layer 21, and one or more secondary layers arranged therebeneath and configured to provide the isolation/integration layer 19 certain additional desired properties, such as moisture seal, electrical insulation, temperature isolation, electromagnetic isolation and/or filtering. Such additional desired properties can be obtained using one or more additives to the one or more secondary layers i.e., the secondary layers can be prepared from the same/similar material used for the main layer but with certain additives for providing the certain additional desired properties. In some embodiments the integration layer 24 is similarly configured as a multilayered structure comprising various different layers having different properties and/or made of different materials for providing certain additional desired properties to the integration layer 24 e.g., by having a main layer configured for direct integration with the object 11, and one or more secondary layers arranged thereon and configured to provide the integration layer 24 certain additional desired properties.

The sensor device 20 is configured in some embodiments to implement a surface/tactile sensor, similar to sensor device 10 of FIG. 1A, forming a sensing sheet by its multilayered structure 25, configured for positioning two sensing straps/patterns S1 and S2 thereof over regions of the multilayered structure 25 covering the cavity/fluid channel 11c, and positioning its two reference straps/patterns D1 and D2 over regions of the multilayered structure 25 that are attached to the base/cap component 11 (non-deformable regions of the multilayered structure 25).

FIG. 2B shows another multilayered surface/tactile sensor structure 20′, which is substantially similar to the multilayered sensor structure 20 shown in FIG. 2A and configured for sensing differential of internal (Pf) and external (Pa) pressures/forces. The base element 26 of the sensor device 20′ comprises a fluid/air cavity/channel 26v sealably covered by the multilayered structure 25, and further comprises one or more fluid-connection passages 26p for fluidly communicating the fluid/air cavity/channel 26v with a fluid/air pipe/reservoir/duct 26c (e.g., of an appliance/consumer product). The sensor configuration 20′ can be used in applications requiring measurement of properties/condition of a fluid substance maintained inside and/or streamed through a pipe/reservoir/duct 26c, such as exemplified in FIG. 3.

Optionally, but in some embodiments preferably, the base element 11 of the sensor device 20 shown in FIGS. 2A and 2B is a portion of an object/appliance/device to which the multilayered structure 25 is attached/coupled, optionally with the deformable external layer 21 attached to the other side of the multilayered structure. The fluid/air cavity/channel 11c can be an integral part of the base/object 11, or alternatively, it may be formed therein before it is attached to the multilayered structure 25. Similarly, the base element 26 of the sensor device 20′ shown in FIG. 2B can be a portion of an object/appliance/device having fluid/air pipe/reservoir/duct 26c, and the fluid/air cavity/channel 26v and the one or more connecting passages 26p can be similarly formed therein before attaching thereto the multilayered structure 25.

For example, FIG. 3 schematically illustrates a sensing catheter device 30 having a plurality of sensing devices 20 or 20′, such as shown in FIG. 2A or FIG. 2B, for measuring one or more metric parameters and/or conditions (generally referred to herein as measured fluid properties or condition) of a fluid substance contained inside or streamed through the tube 31 of the catheter device 30, or of a fluid substance located outside the tube 31 of the catheter device 30 (e.g., a catheter device 30 placed inside the esophagus to measure esophageal mobility, or a finger of a robot with multiple sensing points). Accordingly, in this specific and non-limiting example the fluid/air pipe/reservoir/duct 26c of the sensing devices 20′/20 is implemented by the fluid lumen of the catheter tube 31, in/through which the fluid substance which properties are being measured is contained/streamed.

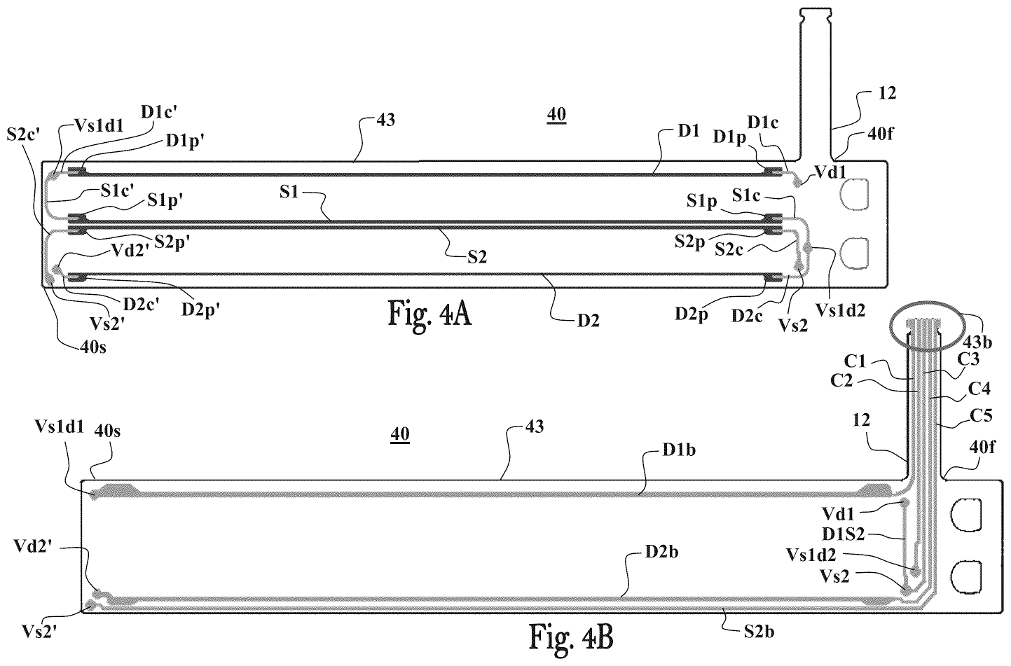

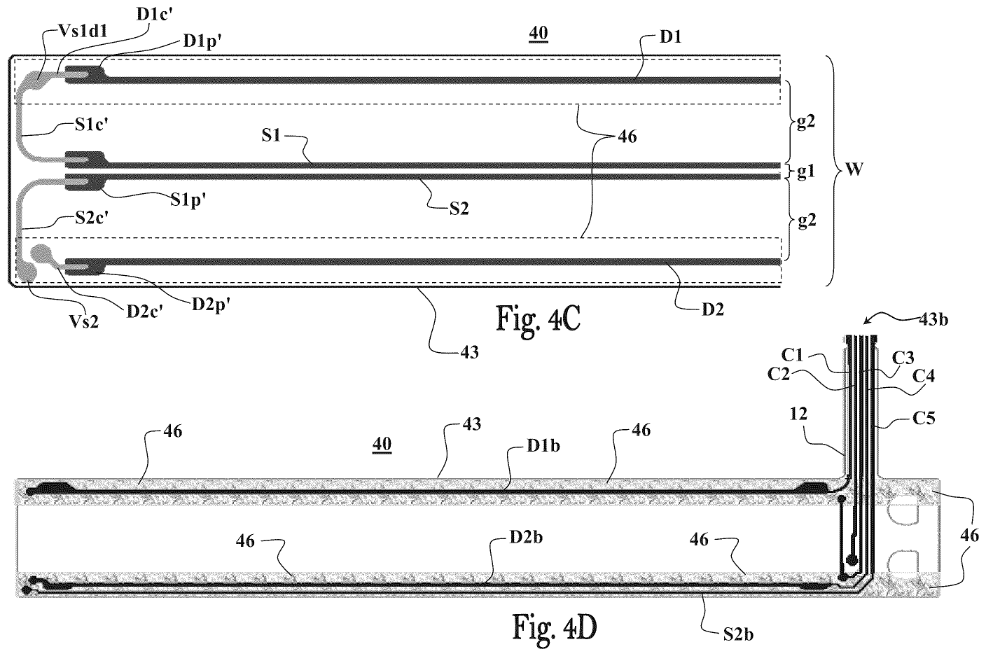

FIG. 4A shows a top view of a sensor device 40 utilizing the configuration of the sensor device 10 shown in FIG. 1A, and having additional connectivity for readily implementing a full/partial bridge circuitry, and an electrical connection arm 12 (e.g., having a flat cable configuration with connector portion 43b extremity) laterally extending from a first end portion 40f of the sensor device 40.

Particularly, the substrate layer 43 of the sensor device 40 comprises at its first end portion 40f the following conductive patterns:
D1c—electrically connecting a contact point D1p at a first end of the reference strap/pattern D1 to an adjacently located via Vd1 e.g., the contact point D1p can be part of the reference strap/pattern D1 i.e., made of a piezoresistive material, and the conductive pattern D1c can be at least partially applied over the contact point D1p, or vice versa;
S1c—electrically connecting a contact point S1p at a first end of the sensing strap/pattern S1 to an adjacently located via Vs1d2 e.g., contact point S1p can be part of the sensing strap/pattern S1 i.e., made of a piezoresistive material, and the conductive pattern S1c can be at least partially applied over the contact point S1p, or vice versa;
S2c—electrically connecting a contact point S2p at a first end of the sensing strap/pattern S2 to an adjacently located via Vs2 e.g., contact point S2p can be part of the sensing strap/pattern S2 i.e., made of a piezoresistive material, and the conductive pattern S2c can be at least partially applied over the contact point S2p, or vice versa; and
D2c—electrically connecting a contact point D2p at a first end of the reference strap/pattern D2 to the via Vs1d2 e.g., contact point D2p can be part of the reference strap/pattern D2 i.e., made of a piezoresistive material, and the conductive pattern D2c can be at least partially applied over the contact point D2p, or vice versa.

The sensor device 40 comprises at its second end portion 40s the following conductive patterns:
D1c′—electrically connecting a contact point D1p′ at a second end of the reference strap/pattern D1 to an adjacently located via Vs1d1 e.g., contact point D1p′ can be part of the reference strap/pattern D1 i.e., made of a piezoresistive material, and the conductive pattern D1c′ can be at least partially applied over the contact point D1p′, or vice versa;
S1c′—electrically connecting a contact point S1p′ at a second end of the sensing strap/pattern S1 to the via Vs1d1 e.g., contact point S1p′ can be part of the sensing strap/pattern S1 i.e., made of a piezoresistive material, and the conductive pattern S1c′ can be at least partially applied over the contact point S1p′, or vice versa;
S2c′—electrically connecting a contact point S2p′ at a second end of the sensing strap/pattern S2 to an adjacently located via Vs2′ e.g., contact point S2p′ can be part of the sensing strap/pattern S2 i.e., made of a piezoresistive material, and the conductive pattern S2c′ can be at least partially applied over the contact point S1p′, or vice versa; and
D2c′—electrically connecting a contact point D2p′ at a second end of the reference strap/pattern D2 to an adjacently located via Vd2′ e.g., contact point D2p′ can be part of the reference strap/pattern D2 i.e., made of a piezoresistive material, and the conductive pattern D2c′ can be at least partially applied over the contact point D2p′, or vice versa.

Optionally, but in some embodiments preferably, at least some portion of the conductive patterns D1c, S1c, S2c, D2c, D1c′, S1c′, S2c′, D2c′ are overlapped by at least some portion of their respective contact points D1p, S1p, S2p, D2p, D1p′, S1p′, S2p′, D2p′ of the straps D1, S1, S2, D2, at their connection areas i.e., at least some portion of these conductive patterns is patterned on their respective contact points, or vice versa.

FIG. 4B shows a bottom side of the sensor device 40, wherein additional conducting patterns are formed to implement the bridge circuit connectivity via the conducting lines C1, C2, C3, C4 and C5, of the electrical connection arm 12. As seen, the via Vs1d2 at the first end portion 40f of the sensor device is directly connected to the conducting line C2 of the connection arm 12, thereby providing electrical connection to the sensing strap S1 and to the reference strap D2 via the conducting line C2, and the via Vs2 at the first end portion 40f of the sensor device is directly connected to the conducting line C3 of the connection arm 12, thereby providing electrical connection to the sensing strap S2 via the conducting line C3. The following conductive patterns are provided at the bottom side of the sensor device 40:
D1b—electrically connecting the via Vs1d1 at the second end portion 40s of the sensor device 40 to the conducting line C1 of the connection arm 12, thereby providing electrical connection to the reference strap D1 via the conducting line C1;
D1S2—electrically connecting between the vias Vd1 and Vs2 at the first end portion 40f of the sensor device 40, thereby providing electrical connection to the reference strap D1 via the conducting line C3 of the connection arm 12;
D2b—electrically connecting the via Vd2′ at the second end portion 40s of the sensor device 40 to the conducting line C4 of the connection arm 12, thereby providing electrical connection to the reference strap D2 via the conducting line C4; and
S2b—electrically connecting the via Vs2′ at the second end portion 40s of the sensor device 40 to the conducting line C5 of the connection arm 12, thereby providing electrical connection to the sensing strap S2 via the conducting line C5.

FIG. 4C and 4D demonstrates attachment of the bottom side of the sensor device 40 to a curved/rounded or flat surface, wherein FIG. 4C shows an extremity of the top view of the sensor device and FIG. 4D a bottom view of the sensor device with two attachment regions 46 (e.g., covered with an adhesive material, or attached by laser welding or any other suitable welding technique) extending along lateral side portions of the bottom side of the sensor device 40.

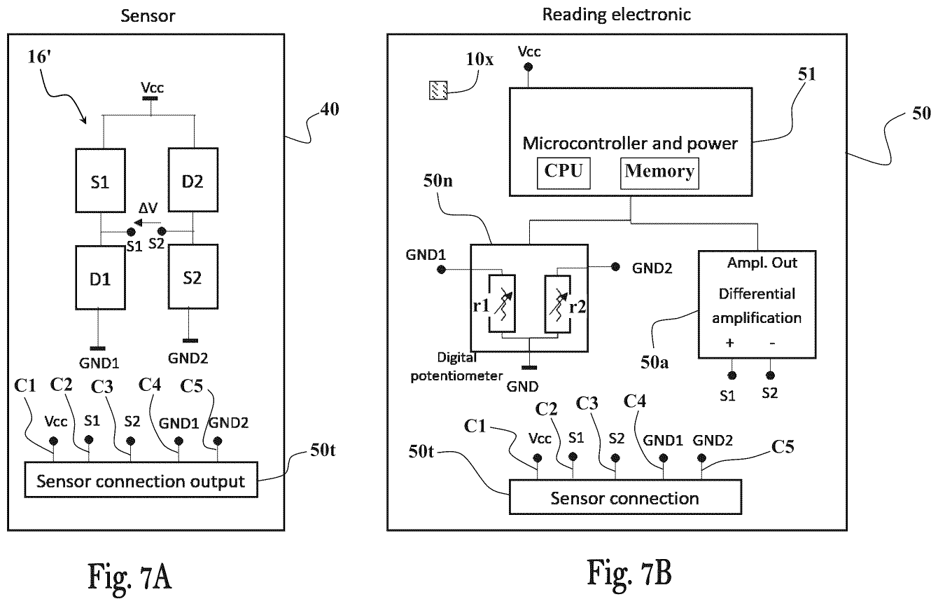

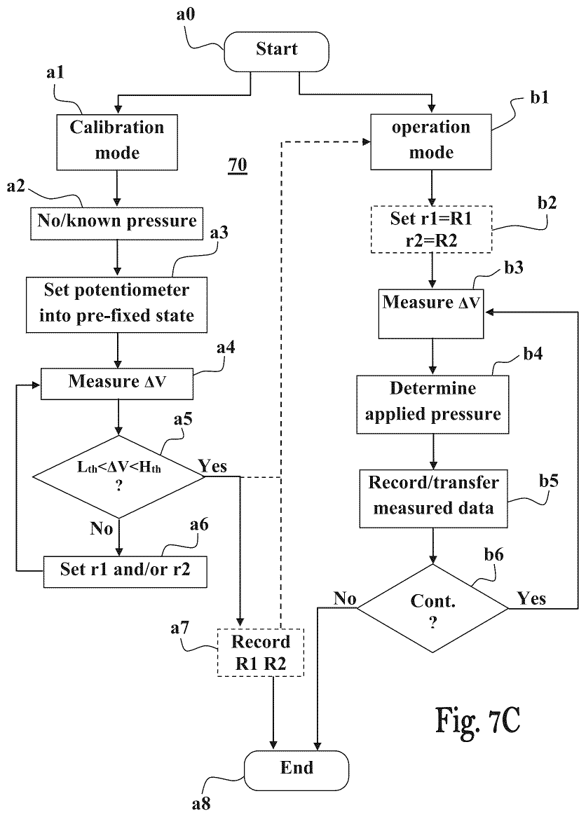

The connectivity provided using the configuration of FIGS. 4A to 4D can be used to construct the following bridge configuration (16′ in FIG. 7A) via the conducting lines C1-C5 of the connection arm 12—

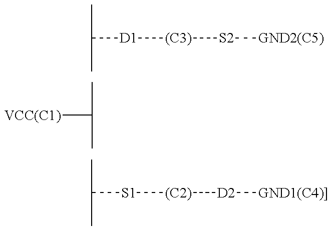

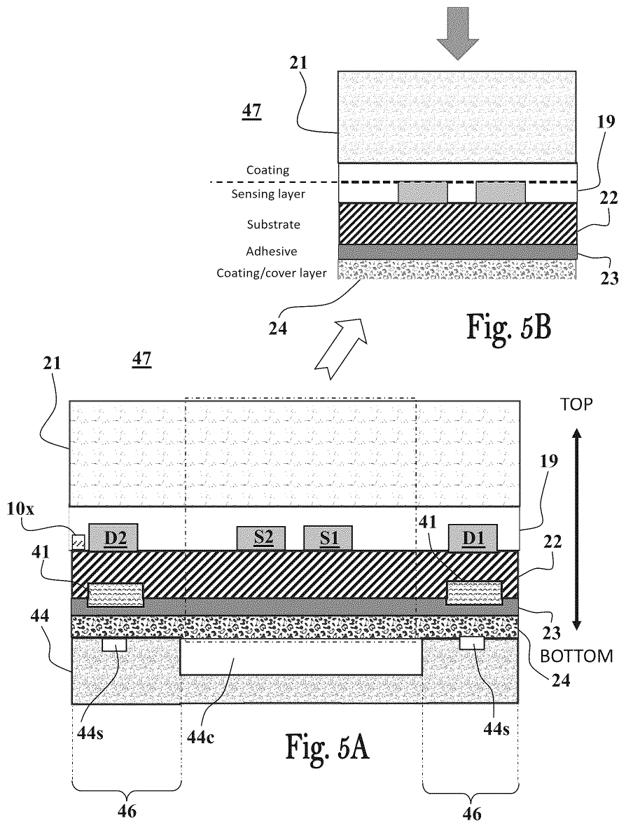

FIGS. 5A and 5B schematically illustrate structure of a multilayered sensor design 47, wherein FIG. 5A shows a sectional view of the sensor device, and FIG. 5B shows various layers of the sensor device 47. As seen, the sensor device 47 is substantially similar to the sensor device 20 shown in FIG. 2A, but further comprises electrically conducting elements 41 formed in/on the bottom side of the substrate layer 22 (e.g., D1b, D2b and/or S2b in FIG. 4B) for providing the connectivity required to implement measurement circuitries (e.g., such as bridge circuitry 16 of FIG. 1C), and its base/object 44 comprises further to the fluid cavity/channel 44c two or more positioning channels 44s covered by the integration layer 24. In possible embodiments at least one, or both, of the reference strap/pattern D1,D2 is formed in/on the bottom side of the substrate layer 22. Similarly, in possible embodiments at least one, or both, of sensing strap/pattern S1,S2 is formed in/on the bottom side of the substrate layer 22.

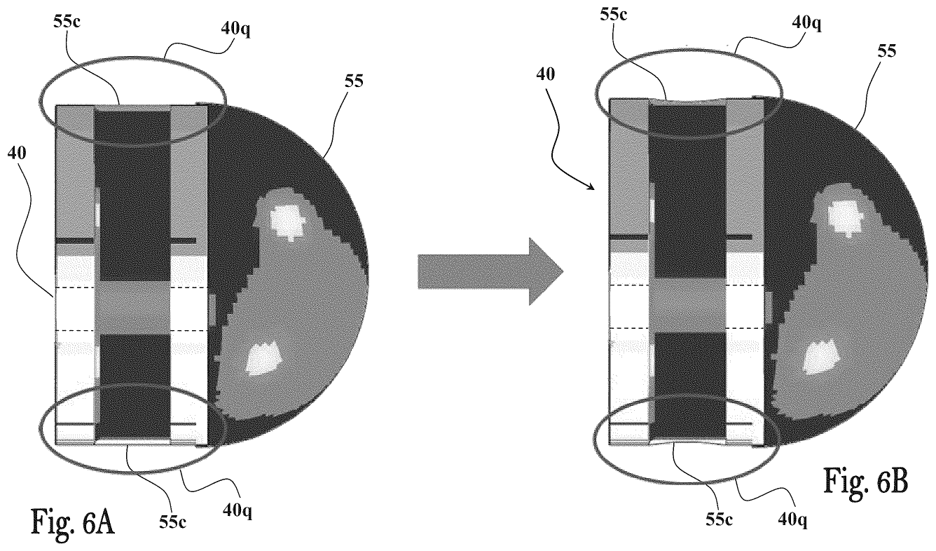

FIG. 6A and 6B show simulation results obtained for the surface/tactile sensor 40 configuration of FIGS. 4 and 5 when applied over a hollow/cylindrical circular object 55 having a circumferential cavity 55c. Accordingly, the sensing straps/patterns (S1,S2 not shown in FIGS. 6A and 6B) are located over the circumferential cavity 55 and thus susceptible to deformations of the sensor substrates in response to pressure/forces applied thereover. The reference straps/patterns (D1,D2 not shown in FIG. 6A and 6B) are located in the attachment regions 46, which are directly connected to wall sections of the circular object 55 i.e., non-deformable portions of the sensor 40. As the sensor 40 is applied over a cylindrical/circular object 55 it assumes a curved/circular configuration, such as shown in FIG. 1D.

In this simulation pressures in the range of 0-200 mBar were exerted on the surface/tactile sensor device 40 to simulate foil deflection, as can be observed at the encircled regions 40q in FIG. 6B. It is noted that FIGS. 6A and 6B do not show the skin/external layer material (21 in FIGS. 2A and 2B) of the sensor device 40. It is further noted that the inventors hereof also manufactured prototypes of the surface/tactile sensor 40 simulated in FIGS. 6A and 6B, and obtained satisfactory tests results, as this simulation predicts.

FIGS. 7A and 7B schematically illustrate interfacing the sensor device 40 to a measuring circuitry 50 for acquiring measurement data/signals therefrom. FIG. 7A exemplifies the connectivity to the bridge circuitry 16′ implemented by the sensing/reference straps (S1, S2)/(D1, D2) of the sensor device 40 via the conducting lines C1, C2, C3, C4 and C5, of the electrical connection arm (12) of the sensor device 40. In this non-limiting example connectivity to the bridge circuitry 16′ is provided via the ports of the sensor connection unit 50t, wherein the source voltage Vcc port is accessible via the conducting line C1 of the connection arm 12, the measurement port s1 is accessible via the conducting line C2 of the connection arm 12, the measurement port s2 is accessible via the conducting line C3 of the connection arm 12, the first ground port GND1 is accessible via the conducting line C4 of the connection arm 12, and the second ground port GND2 is accessible via the conducting line C5 of the connection arm 12.

FIG. 7B shows a possible measurement circuitry 50 with automatic offset compensation for the bridge circuitry 16′ shown in FIG. 7A. In this specific and non-limiting example, the measurement circuitry 50 for bridge circuitry 16′ comprises a control and power unit 51, a digital potentiometer unit 50n, an amplification unit 50a, and sensor connectivity unit 50t for electrically connecting to the various ports of the bridge circuitry 16′. The amplification unit 50a is configured to amplify (e.g., utilizing a differential amplifier) the voltage signals ΔV measured between the signal ports s1 and s2 of the bridge circuitry 16′, as obtained via the C2 and C3 conducting lines of the electrical connection arm 12. The digital potentiometer unit 50n comprises one or more controllably variable electrical resistor elements r1, r2, . . . electrically connected to the ground ports GND1, GND2, . . . , respectively.

The control and power unit 51 is configured to energize the measurement circuitry 50 and/or the sensor device 40, receive and process the amplified measurement signals from the amplification unit 50a and generate measurement data indicative thereof, and calibrate the bridge circuitry 16′ by setting the one or more controllable variable electrical resistor elements r1, r2, . . . of the digital potentiometer unit 50n.

The digital potentiometer unit 50n shown in FIG. 7B comprises two controllable variable electrical resistor elements r1 and r2 electrically connected to the ground ports GND1 and GND2, respectively. However, in possible embodiments, a single controllable variable electrical resistor element, r1 or r2, is used to calibrate the bridge circuitry 16′. The measurement circuitry 50 can be an external unit electrically connected to the sensor device 40, but in some possible embodiments it is embedded as an integral part of the sensor device 40 e.g., on (or in) the substrate layer 22. In alternative embodiments, the bridge circuitry 16′ is arranged to provide Vcc1 and Vcc2 voltage supply ports to the (S1,D1) and (D2,S2) voltage dividing branches, respectively, and a single GND ground port at the other end of these voltage dividing branches (instead of the GND1 and GND2 ground ports), and in such embodiments the digital potentiometer unit 50n can be configured to connect to the Vcc1 and Vcc2 voltage supply ports of the voltage dividing branches.

FIG. 7C demonstrates possible calibration and measurement schemes 70 according to some possible embodiments. The control unit (51) can be configured to change between calibration (a1) and operation (b1) modes after its operation starts (a0). If calibration (a1) is required, the sensor device (40) is operated in a rest/no-pressure (or under a known pressure) state (a2), the controllable variable electrical resistor elements r1 and r2 are set to a pre-fixed value (a3 e.g., half of their maximal resistance value), and the control unit 51 acquires (a4) the measurement signal AV amplified by the amplification unit (50a). The control unit 51 then checks (a5) if the measured signal AV obtained is between predetermined high (Hₜₕ) and low (Lₜₕ) expected threshold voltage values.

If the measured signal ΔV is not within the high (Hₜₕ) and low (Lₜₕ) expected threshold voltage values, the control unit 51 adjusts the value of at least one of the controllable variable electrical resistor elements r1 and r2 by some predefined Δr resistance calibration step, and the control is passed back to the measurement step (a4) to acquire a new measurement signal ΔV from the amplification unit (50a). For example, if it is determined from the acquired measurement signal ΔV that the voltage over port s1 is higher than then voltage over port s2 (e.g., the amplified voltage is higher than a defined reference voltage, for example 0V), then the control unit 51 may increase the value of r2 until the voltage over port s2 equals (e.g., within a defined tolerable error) to the voltage over s1.

If it is determined (in step a5) that the measured signal ΔV equals zero/negligibly small, then the values of R1 and R2 of the controllable variable electrical resistor elements r1 and r2, respectively are used in the operation mode (b1), and optionally stored (marked by dashed box a7) in the memory of the control unit 51. The process can be stopped (a8) after the calibration process, or alternatively, change into the operation mode (b1) for reading measurement signals from the sensor device (40). This way the sensor device (40) can be calibrated without any trimming elements.

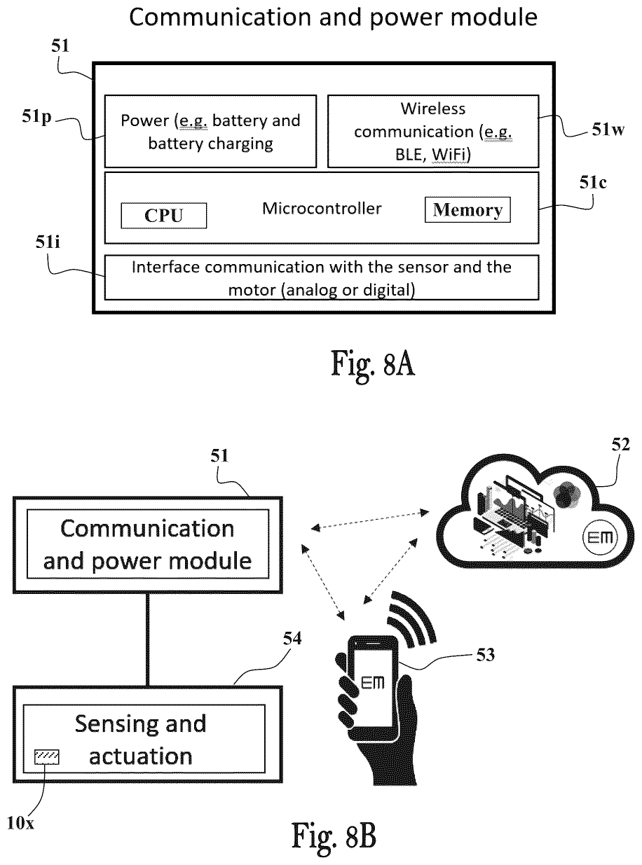

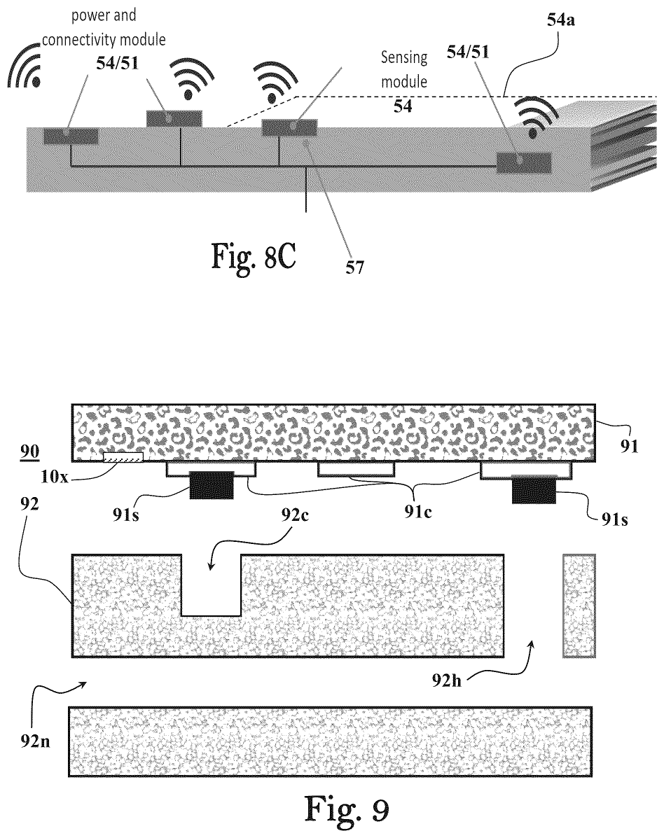

When changed into the operation mode (b1), the control unit (51) optionally sets (if not previously set during the calibration process) the values of the controllable variable electrical resistor elements r1 and r2 to the R1 and R2 values respectively stored in its memory (b2). The control unit (51) can then acquire one or more measurement signals ΔV (b3) from the amplification unit (50a), process the measured signals (b4) to determine based thereon the applied pressure/force and generate measurement data/signals indicative thereof, and store the generated measurement data/signals in its memory and/or transfer the same to external/other devices (as exemplified in FIG. 8A to 8C) for further processing/operations (b5). The operation mode can be stopped (a8) if it is determined that there is no need for further measurement data/signals ΔV (b6).

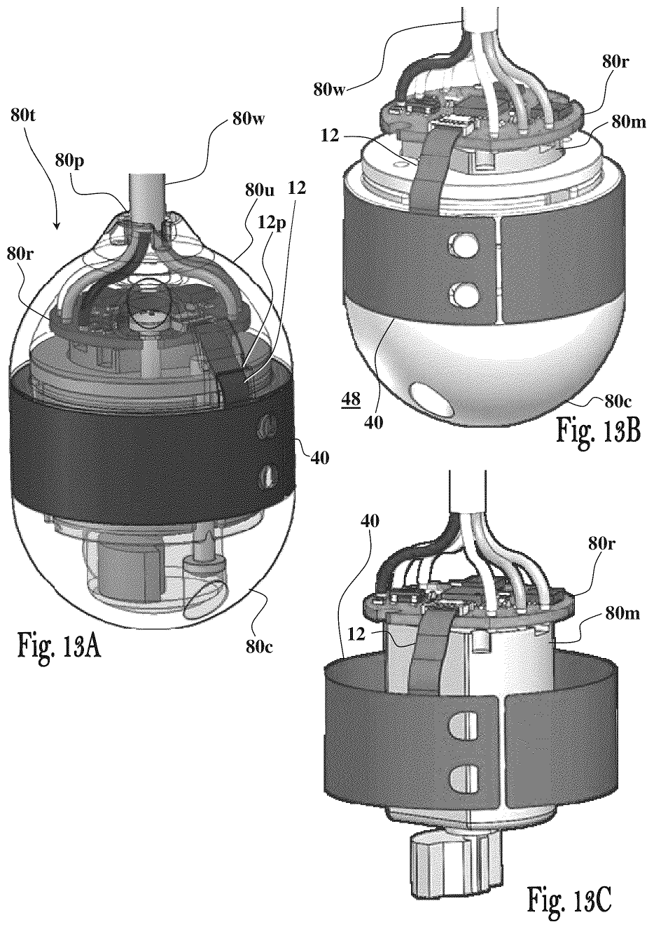

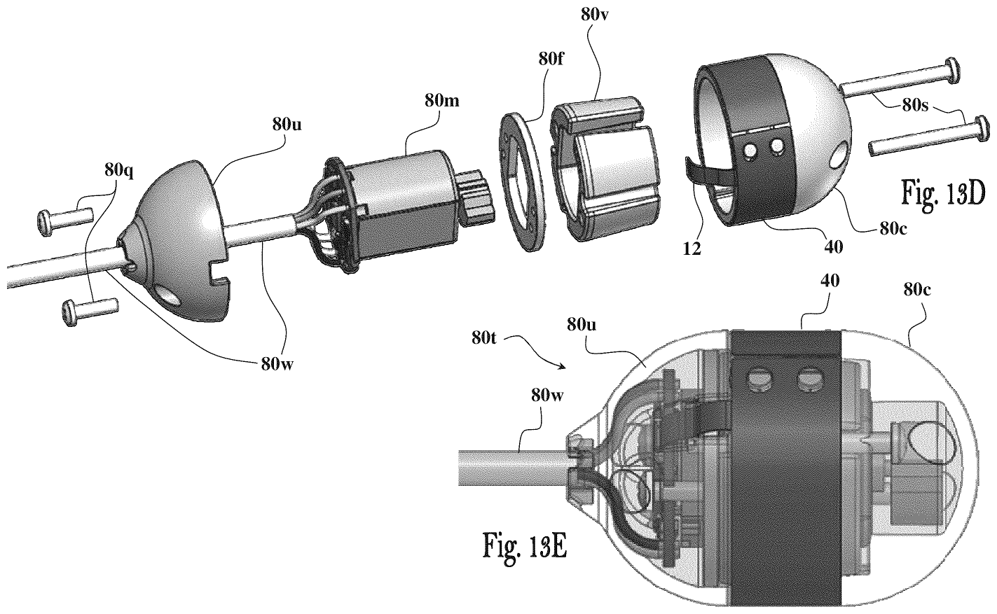

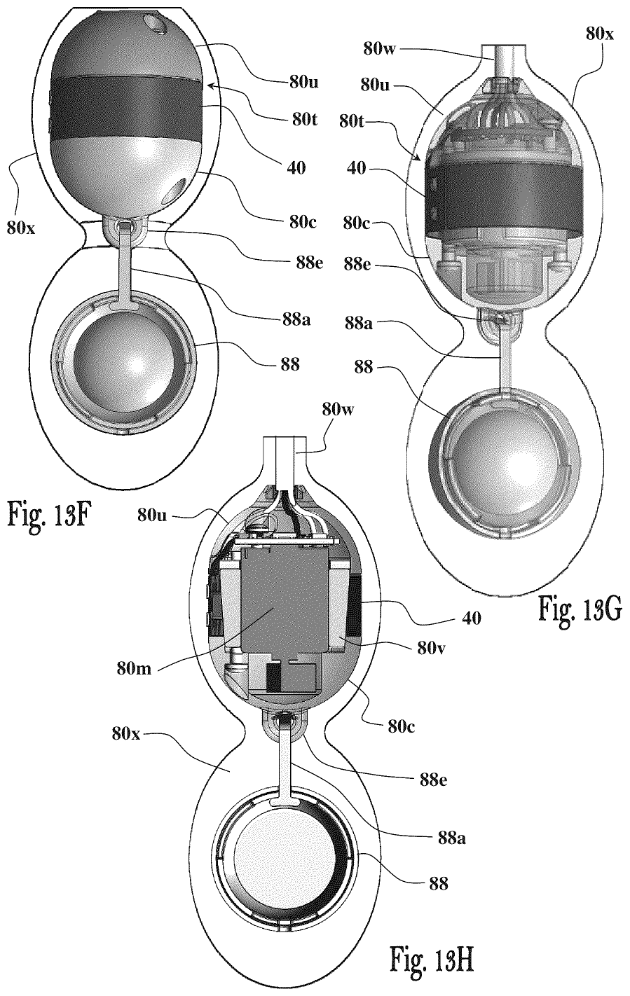

FIG. 8A shows a possible communication and power module 51 usable for the sensor devices disclosed herein. In this non-limiting example the communication and power module 51 comprises a power source (e.g., battery/charger) 51p, a data communication unit 51w (e.g., over serial/parallel data bus/wires and/or wireless data communication channel, such as, but not limited to, WiFi, Bluetooth/low energy-BLE, or suchlike), one or more processors and memories 51c, and an interfacing (analog or digital) module 51i configure to acquire measurement data/signals from the sensor device(s) and/or activate other devices (e.g., actuator(s) and/or motor as exemplified in FIGS. 13A to 13H).

FIG. 8B demonstrates communication of measurement data/signals acquired from the sensor device according to possible embodiments. In this specific and non-limiting example the control and power unit 51 is configured to acquire measurement data/signals from one or more sensor devices (e.g., according to any of the embodiments disclosed herein) and/or generate control data/signals for operating one or more devices (e.g., actuator/electric motor) of a sensing and actuation unit 54. The control and power unit 51 can be configured to wirelessly communicate data/signals with an external device 53 e.g., a smart device, such as a smartphone, tablet, PDA, PC, or suchlike. The external device 53 can be configured to run one or more applications configured to receive and process the data/signals transmitted from the control and power unit 51, and transmit the same to the control and power unit 51 data/signals for acquisition of further measurement data/signals and/or operation of the one or more devices of the sensing and actuation unit 54.

Optionally, but in some embodiments preferably, external device 53 is configured to communicate data/signals with a remote computer/server (e.g., database repository, control center, cloud) 52 for further processing and/or recording the same therein e.g., over one or more data networks (e.g., the Internet). Alternatively, or additionally, the control and power unit 51 can be configured to communicate data/signals directly with the remote computer/server 52. The control and power unit 51 and the sensing and actuation unit 54 can be implemented utilizing any of the embodiments disclosed and claimed in International Patent Publication No. WO 2022/149128 titled “modular sensor designs and applications thereof”, of the same applicant hereof, the disclosure of which is incorporated herein by reference.

FIG. 8C demonstrates application of multiple surface/tactile sensing devices 54 and power/connectivity units 51 in a sensing structure (e.g., synthetic/sensing skin) 57 configured for sensing over continuous wide surface areas 54a. As seen, the sensing device 54 can have a multilayered structure implemented as disclosed herein for integration in (e.g., just below the surface or part of the surface) or on the sensing structure 57. Each sensor device 54 can be electrically connected to a respective control and communication unit 51 configured to acquire the measurement data/signals thereof and transmit the same to an external/remote device/system (53/52 in FIG. 8B).

The measurement data/signals acquired from the sensor devices 54 can be used for biofeedback and control of the actuator/motor(s) speed and cycles, as applicable to the embodiments shown in FIGS. 11 to 13.

FIG. 9 schematically illustrates a sensor design 90 according to possible embodiments assembled as a multilayer structure comprising at least two layers: an isolation layer 91 (e.g., thin film/foil) having electrical, thermal and/or humidity isolation properties, which may further serve as an integration layer e.g., for attachment, and an electrically conducting layer 91c applied/patterned on the isolation layer 91. The conductive and/or gauging tracks 91c formed/patterned on the isolation layer 91 can be configured to electrically connect to/between sensor elements 91s, and/or additional elements assembled on/to the isolation layer 91 (e.g., integrated circuits, passive devices, ASICS, etc.). Alternatively, or additionally, the conductive and/or gauging tracks 91c can be configured to implement one or more antenna elements, electrical connection pads and/or circuitries, etc.

The sensor device 90 can be attached to a base body/object 92 e.g., made from a plastic/polymer material, having one or more cavities 92c, each configured to accommodate at least one of the sensor elements 91s. In possible embodiments the base body 92 may have one or more fluid channels 92n configured to maintain/stream a fluid substance therethrough, and one or more fluid passages 92h in fluid communication with at least one of the one or more fluid channels 92n and configured to at accommodate at least partially at least one of the sensor elements 91s. The sensor elements 91s accommodated inside the one or more cavities 92c can be configured for contactless measurement one or more properties and/or conditions of a fluid substance maintained/streamed inside the one or more fluid channels 92n. The one or more sensor elements 91s accommodated in the fluid passages 92h can be configured to interact with the fluid substance maintained/streamed inside the one or more fluid channels 92n by direct contact therewith, and/or responsive to deformations occurring in the isolation layer 91.

The isolation and integration layer 91 can be configured to isolate and protect the sensor elements 91s from the external environment, and/or allow the integration of the sensor device 90 with the base body/object 92 (e.g., by heat welding, laser welding, ultrasonic welding, overmolding, etc.)

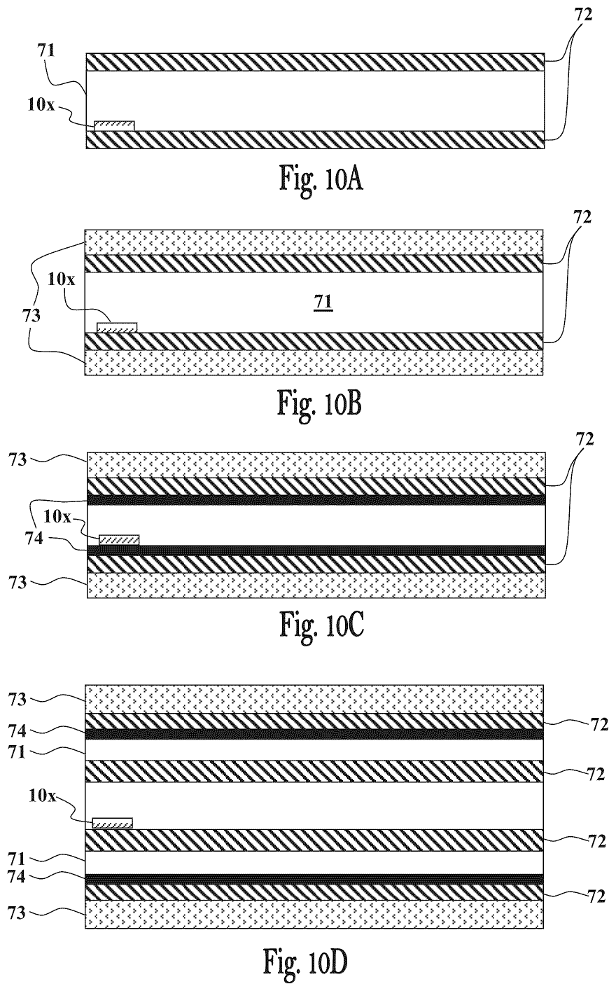

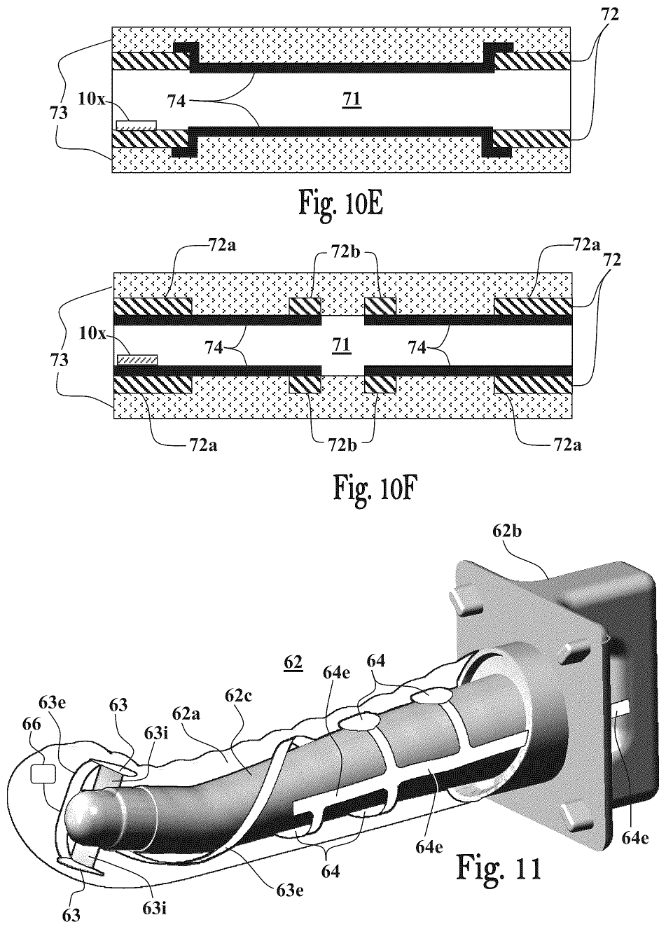

FIGS. 10A to 10F are sectional views of possible multilayered sensor structures. FIG. 10A shows an isolation/integration layer/substrate 71 (e.g., flexible/elastic or stiff thin film/foil, or a thick flexible/elastic or stiff sheet of material) having two electrically conducting layers 72 applied/patterned over two opposite side surfaces thereof. At least one of the electrically conducting layers 72 is configured to accommodate one or more sensor devices (not shown) according to any of the embodiments disclosed, or incorporated by references, herein.

FIG. 10B shows an isolation/integration layer/substrate 71 having the two electrically conducting layers 72 applied over its two opposite side surfaces, and two protective/integration layers 73, wherein each protective layer 73 is applied over a respective one of the electrically conducting layers 72. At least one of the electrically conducting layers 72 is configured to accommodate one or more sensor devices (not shown) according to any of the embodiments disclosed, or incorporated by references, herein.

FIG. 10C shows the layered structure of FIG. 10B with sensing/gauging layers (e.g., piezoresistive, piezoelectric, etc.) 74 sandwiched between the isolation/integration layer/substrate 71 and the electrically conducting layers 72 (at least some portion of the electrically conducting layer 72 located on top the sensing layers 74 can be removed utilizing any suitable technique, such as described in the International Patent Publications indicated hereinabove). At least one of the electrically conducting layers 72 and/or the sensing layers 74 is configured to accommodate one or more sensor devices (not shown) according to any of the embodiments disclosed, or incorporated by references, herein.

FIG. 10D shows the layered structure of FIG. 10C with multiple electrically conducting layers 72 and isolation/integration layers 72 configured to form a multilayered sensing structure similar to a multilayer PCB structure e.g., have buried sensors and/or connections lines. At least some of the electrically conducting layers 72 can be electrically connected by vias (not shown). At least one of the electrically conducting layers 72 and/or the sensing/gauging layers 74 is configured to accommodate one or more sensor devices (not shown) according to any of the embodiments disclosed, or incorporated by references, herein.

The electrically conducting layers 72 and/or the sensing/gauging layers 74 can be pattered to create tracks and/or any other desired structures/pattern (e.g., RF antennas). Optionally, the protective/isolation/integration layers 73 comprises openings (not shown) configured to allow access to the electrically conducting layer located therebeneath, for components assembly and/or for creating contact pads and/or electrodes. Additional protective and/or conductive layers 73 can be applied over the exposed electrically conducting (e.g., gold, platinum, etc.) layers 72. Optionally, but in some embodiments preferably, the protective/isolation/integration layers 73 is a multilayered structure comprising various different layers having different properties and/or made of different materials for providing certain additional desired properties to the protective/isolation/integration layers 73 e.g., by having a main layer configured for direct integration with an object, and one or more secondary layers configured to provide the additional desired properties.

Optionally, the protective/isolation layers 73 and the electrically conducting layers 72 are provided with openings configured for access and passage to the sensing/gauging layers 74 located therebeneath, and/or to provide the sensing layer 74 direct contact with the carrying object (e.g., 92 in FIG. 9) or the external environment, or in correspondence with one of the cavities/openings of the carrying object. In possible embodiments at least one of the sensing layers 74 is configured in direct contact with at least one of the protective layers/isolation/integration 73. In some applications the different layers of these multilayered structures are laminated with one or more adhesive (not shown) or adhesive-less layers.

FIG. 10E exemplifies a possible sensor constructions scheme wherein the sensing layer 74 is applied/deposited after etching at least some portion of the electrically conducting layer 72, or after the deposition of at least some portion of the electrically conducting layer 72. For example, the production of the sensor of FIG. 10E can start with preparation of the layered structure shown in FIG. 10A, which is then etched to remove some portion(s) of the formed (or selectively deposited) electrically conducting layers 72, proceed with application/patterning of the sensing layers 74 over portions of the isolation/integration layer/substrate 71 from which the electrically conducting layers 72 has been removed (or selectively applied), and can be finalized in application of the protective layers 73. As exemplified in FIG. 10E, the sensing layers 74 can be applied/patterned at least partially over portions of the electrically conducting layers 72 to establish reliable electrical connection therebetween.

FIG. 10F exemplifies a possible modification of the sensor device of FIG. 10C, wherein the sensing layers 74 are patterned/etched after they are applied/patterned on the isolation/integration layer/substrate 71, and the electrically conducting layers 72 are selectively patterned on the sensing layers 74, or etched to remove some portions thereof after they are applied/patterned on the sensing layers 74. Finally, the protective/isolation/integration layers 73 are applied over the electrically conducting layers 72 and/or the sensing layers 74. Accordingly, in this non-limiting example the electrical contact between the sensing layers 74 and the conducting layers 72 is established by placing at least some portion (e.g., 72a and 72b) of the electrically conducting layers 72 over the sensing layers 74.

FIG. 11 schematically illustrates a device 62 configured for practicing sphincter (annular) muscles, and/or measure application of pressure in specific/discrete points/locations therealong e.g., to measure tactile pressure/force in specific points along a bionic finger. The device 62 comprises a rigid and/or stiff, or semirigid (e.g., made from flexible or semirigid plastic material) core structure 62c distally extending from a rigid/semirigid base element 62b. Optionally, but in some embodiments preferably, the core structure 62c distally tapers as it extends away from the base element 62b, and at least some portion thereof has a cylindrical/frustoconical geometric shape. The core structure 62c is covered by one or more flexible/soft layers (e.g., made from rubber, silicon) 62a in which a plurality of sensor elements 63 and 64 are embedded. The embedded sensor elements 63 and 64 can be positioned inside/in-between the one or more flexible/soft layers 62a and/or on its outer surface.

The device 62 can have a plurality of sensor elements 64 distributed along some portion of the length of the core structure 62c and optionally about circumferences thereof, and a plurality of sensor elements 63 distributed about a circumference of a distal tip region of the core structure 62c. In this specific and non-limiting example four sensor elements 64 are positioned along the length of the core structure 62c, wherein two of the sensor elements 64 are positioned above the core structure 62c and other two of the sensor elements 64 are positioned beneath the core structure 62c. The sensor elements 64 are electrically connected to a data/signal bus of electrical conductors 64e e.g., flexible (optionally non-stretchable) foil with embedded conductors. Similarly, the plurality of (in this example two) sensor elements 63, are electrically connected to a data/signal bus of electrical conductors 63e e.g., flexible (optionally non-stretchable) foil with embedded conductors. In this specific example the data/signal bus 63e helically extend along a length of the core structure 62c to for increased stretchability.

In some embodiments the distal tip portion of the device 62 comprises an accelerometer sensing module 66 configured to measure vibrations and/or movements in the device 62 e.g., that cannot be sensed by the sensor elements 63 and/or 64. As seen, the accelerometer sensing module 66 can be electrically connected to the data/signals bus 63e. The sensor elements 63 and/or 64 can be implemented using any of the sensor device embodiments disclosed and/or incorporated by reference herein.

Optionally, but in some embodiments preferably, each of the sensor elements 63 distributed about the circumference of the distal tip region of the core structure 62c is placed over a respective cavity 63i configured to facilitate deformations of the sensor elements in response to externally applied forces/pressures. In use, the device 62 can be introduced into the body of a patient (e.g., vagina) for sensing forces/pressures applied thereover by sphincter (annular) muscles of a treated/exercised subject. The measurement data acquired by the plurality of sensor devices 63,64 can be presented to the treated/exercised subject and/or a practitioner for performance evaluation and/or feedback.

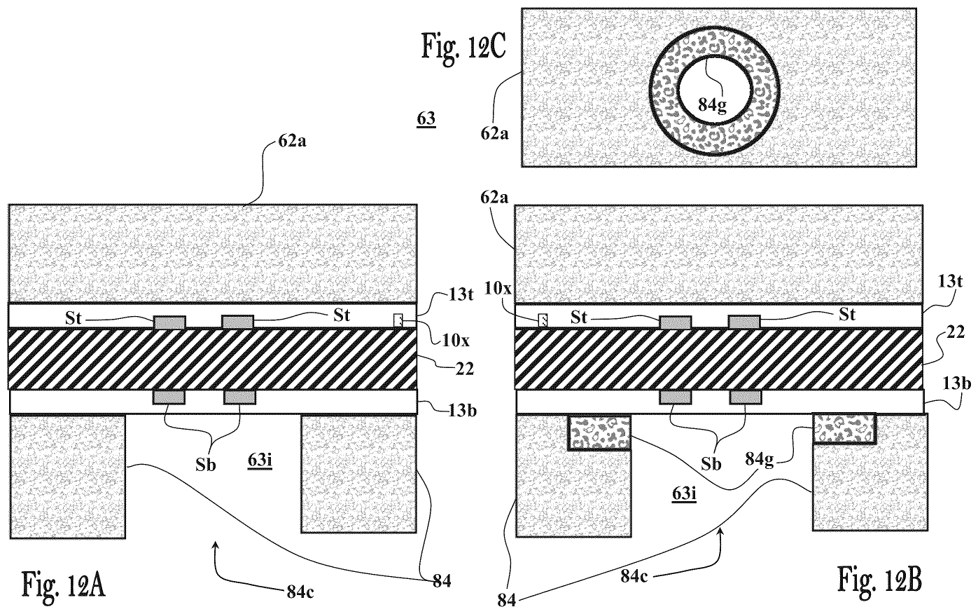

FIGS. 12A to 12C schematically illustrate another sensor configuration according to some possible embodiments suitable for implementing the sensor devices 63 distributed about the circumference of the distal tip of the core structure 62c of the device 62 shown in FIG. 11. The sensor device 63 comprises a substrate 22 having one or more sensor elements St,Sb formed/patterned on (or in) its top and bottom sides, and top and bottom integration layers 13t,13b e.g., made of polyimide (e.g., Kapton), PEEK, fiber reinforced composite materials, PET, PC, Silicone, Rubber, or any other suitable polymeric material, applied to cover the substrate and their sensing elements St,Sb. In possible embodiments the one or more sensor elements St,Sb can be formed/patterned on (or in) one side of the substrate 22 i.e., either on its top or bottom side. The sensor device 63 comprises one or more flexible/soft layers 62a,84 covering at least some portion of its outermost top/bottom integration layers 13t,13b. Optionally, but in some embodiments preferably, a cavity/opening 63i is formed in at least one of the flexible/soft layers 62a,84 to facilitate deformations of portions 84c of the sensor device on/in which the sensor elements St,Sb are formed/patterned.

As shown in FIGS. 12B and 12C, the sensor device 63 comprises in some embodiments a reinforcing/stiff rigid (e.g., annular) element 84g configured to at least partially encircle/surround the cavity 63i over which the sensor elements St,Sb are located, to thereby provide support to the sensor device 63 while enabling deformations of the portions 84c of the sensor device on/in which the sensor elements St,Sb are formed/patterned.

FIGS. 13A to 13H schematically illustrate a practice device 48 configured for practicing sphincter (annular) muscles of a user e.g., Kegel ball. FIGS. 13A to 13E show structure and components of an actuatable device portion 80t of the practice device 48 generally having a three-dimensional ellipsoid/egg-like geometrical shape. The actuatable portion of the device 48 comprises an actuator (e.g., electrical motor) 80m, and a driving and communication circuitry 80r electrically coupled thereto, which are enclosed inside a hollow housing 80t. The hollow housing 80t is constructed from a sensor shell/cap-shaped structure 80c carrying the sensor device 40 and configured to sealingly connect to a respective motor shell/cap-shaped structure 80u. The diameter of the housing/actuatable portion 80t can be in the range of 25 to 30 millimeters, optionally about 27 millimeters, and its length can be in the range of 30 to 40 millimeters, optionally about 35 millimeters.

The sensor and/or motor shell/cap-shaped structures 80c and/or 80u can be made from a type of rigid material e.g., plastic such as PC, ABS, Nylon or such alike (preferably thermoplastic). The actuator 80m can be implemented using a type of electric motor (e.g., any kind of electric motor, such as a DC motor) configured to vibrate the actuatable portion 80t e.g., at frequencies in the range 0.1 to 5000 Hz. In this non-limiting example, the sensor device 40 is arranged to form a circumferential sensor configuration (such as shown in FIG. 1D) encircling substantially an external annular portion of the sensor shell/cap-shaped structure 80c. The sensor shell/cap-shaped structure 80c can be configured with a circumferential groove configured to accommodate the sensor device 40 and/or facilitate deformations of portions thereof wherein the sensing straps/patterns are located.

The electrical connection arm 12 extends upwardly from the sensor device 40, and it is introduced into the hollow housing 80t via a lateral opening 12p formed in the sensor shell/cap-shaped structure 80c to electrically connect thereinside to the driving and communication circuitry 80r.

An electric cable 80w electrically connected to the driving and communication circuitry 80r can be used to power the driving circuitry 80r and communicate measurement signals/data and/or control signals/data with the device 48 e.g., utilizing two wires for driving the actuator/motor 80m, two wires for communicating the measurement data from the sensor device 40 (e.g., utilizing I2C communication bus/protocol), and two wires for powering the logic/driving and communication circuitry 80r. The electric cable 80w can pass into the hollow housing through an opening 80p formed in an apex of the lid shell/cap-shaped structure 80u.

In possible embodiments the hollow housing 80t may comprise an internal power source (e.g., battery-not shown) and a wireless communication unit (e.g., WiFi, BLE-not shown), configured for powering the driving and communication circuitry 80r and wirelessly communicate the measurement signals/data and/or the control signals/data with the device 48. The wireless communication unit can be installed on the free end of the electrical cable 80w to power the logic/driving and communication circuitry 80r, the sensor 40, the motor 80m, and for communicating thereover e.g., by I2C (addressable device). Optionally, in some embodiments, multiple ball device 48 are serially coupled and electrically connected one to the other to share the same data/signals bus implemented by using of the electrical cable 80w (e.g., as exemplified in FIG. 8C).

FIG. 13D shows an exploded view of the housing/actuatable portion 80t further including a support structure 80v and a fixing flange 80f accommodated inside the hollow housing 80t and configured to hold the actuator/motor 80m in place thereinside. Fixing screws 80s and 80q can be used to connect the sensor and motor shell/cap-shaped structures 80c,80u to the support structure 80v. FIG. 13E shows a side view of the actuatable device portion 80t.

FIGS. 13F to 13H show the practice device comprising a rattling ball 88 coupled to the hollow housing 80t, and encased inside one or more layers of flexible/deformable (e.g., rubber, liquid silicon, or any other suitable silicon materials) coating 80x. The rattling ball 88 is movably attached to the actuatable device portion 80t via a suspension arm 88a connected at one end thereof to the rattling ball 88, and which other end is movably engaged in an eyelet 88e provided in the apex of the sensor shell/cap-shaped structure 80c.

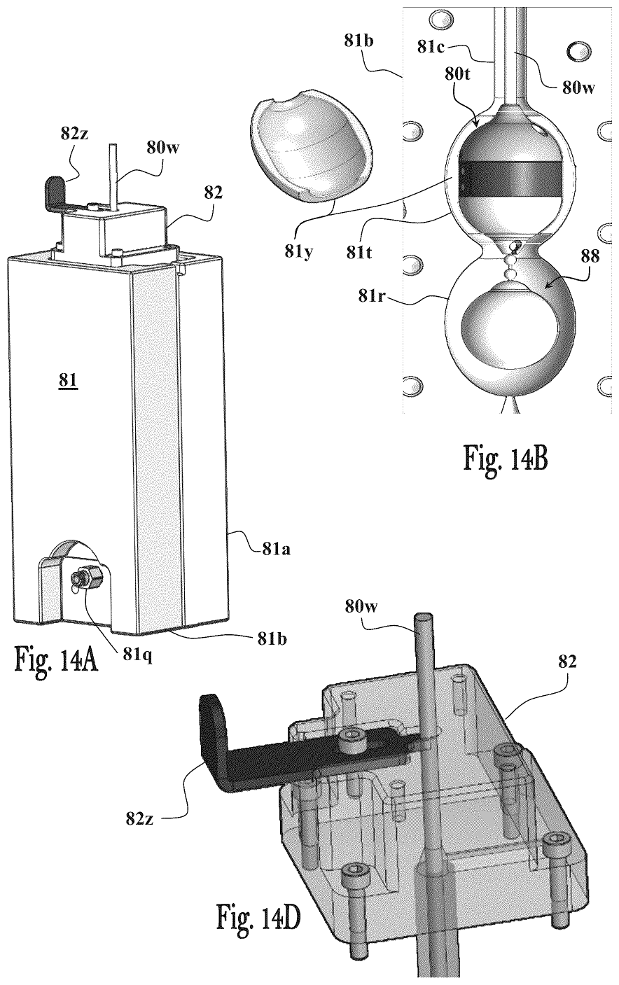

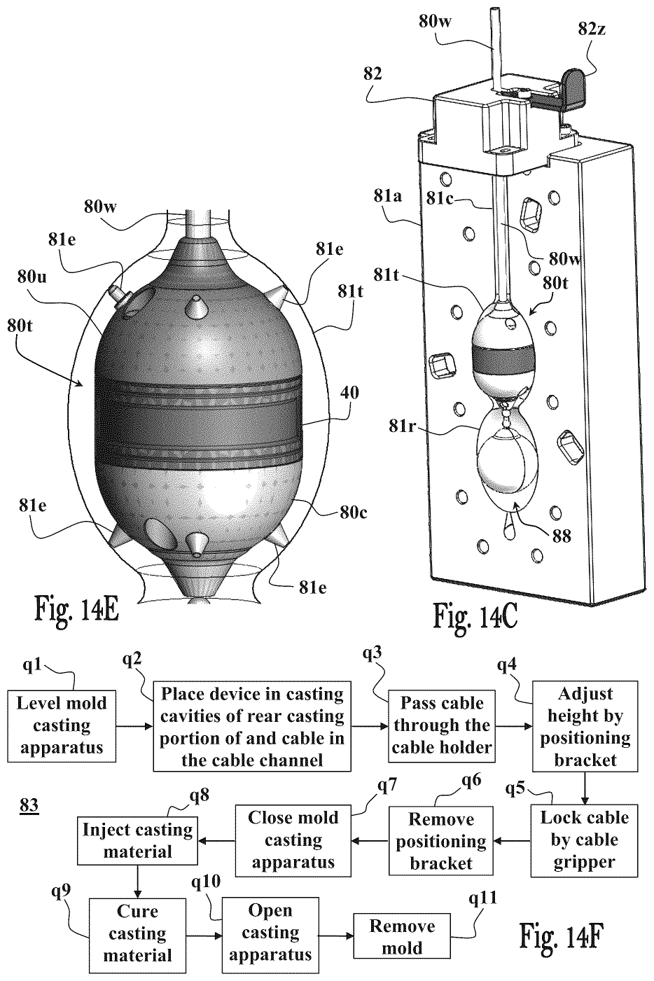

FIGS. 14A to 14E schematically illustrate a casting process and equipment for applying the flexible/deformable enclosure 80x over the components of the practice device. FIG. 14A shows a casting mold apparatus 81 comprising a front portion 81b and a rear portion 81a sealingly attached one to the other, and a cable holder 82 at the upper side thereof. FIGS. 14B and 14C are sectional views of the mold casting apparatus 81, showing the rear portion 81a with the actuatable device portion 80t and the rattling ball 88 readily positioned in respective cavities, 81t and 81r, for mold casting. The front and rear portions 81a,81b of the casting mold apparatus 81 can be configured to define a cable channel 81c extending upwardly from the top apex of the cavity 81t for mold casting at least some portion of the electric cable 80w of the practice device.

FIG. 14E demonstrates use of spacer elements 81e for centering portions of the practice device in their respective mold casting cavities. For example, the actuatable device portion 80t can be centered in its mold casting cavity 81t using a plurality of the spacer elements 81e evenly distributed about one or more circumferential portions of the device 80t. In this specific and non-limiting example two circumferential portions of the device 80t are provided with four evenly distributed spacer elements 81e, at upper and lower portions of the device. The spacers 81e are configured to immobilize the device 80t a defined distance from wall sections of the mold casting cavity 81t, substantially centered thereinside.

The rattling ball 88 can be similarly centered inside its mold casting cavity 81r by a plurality of such spacer elements 81e. However, in possible embodiments, proper design of the suspension arm 88a and eyelet 88e guarantees that the rattling ball 88 is maintained centered inside its mold casting cavity 81r without using spacer elements 81e, upon centering the actuatable device portion 80t in its mold casting cavity 81t by the spacer elements 81e.

FIG. 14F is a flowchart illustrating steps of the mold casting process 83. The process 83 starts in leveling (q1) the rear portion (81a) of the mold casting apparatus (81) e.g., by placement on a leveled surface, and thereafter placing (q2) the actuatable device portion (80t) and the rattling ball (88) inside their respective casting cavities (81t and 81r), and the cable (80w) inside the cable casting channel (81c). Next, the cable (80w) is passed (q3) through the passage of the cable holder unit (82) and the height of the actuatable device portion 80t is adjusted/centered (q4) by the positioning bracket (81y in FIG. 14B e.g., instead of, or in addition to, the spacer elements 81e). After the actuatable device portion (80t) is positioned/centered inside its molding cavity (81t), the cable (80w) is locked (q5) by changing the state of a gripper element (82z) into its locked state, thereby immobilizing the cable (80w) substantially centered inside the cable channel (82c).

The positioning bracket (81y) is then removed (q6), and the mold casting apparatus (81) is then closed (q7) by sealably attaching its front portion (81b) to its rear portion (81a). The casting material (e.g., rubber, liquid Silicon or any other suitable silicon materials) is then injected (q8) into the casting cavities (81t and 81r) via the injection port (81q) provided in the front portion (81b) of the apparatus (81). The casting material can be injected from the bottom of the apparatus (81), as exemplified in FIG. 14A, to facilitate air discharge therefrom e.g., the top side of the apparatus (81) can be provided with a suitable opening configured to facilitate the air discharge and lower the pressure inside the cavities (81t and 81r). The injected casting material can be then cured (q9 e.g., temperature and/or time curing, and any post curing, if needed). After the casting material is cured, the casting apparatus (81) is opened (q10) and the molded practice device is removed (q11) from the casting apparatus (81).

In addition to the force/pressure measurements, the sensor embodiments disclosed can be configured to measure other properties/parameters, such as, but not limited to, temperature, electrical conductivity, optical transmittance and/or reflectance of a medium/tissue. For example, the electrical resistance of one or more of the gauging straps/patterns D1, S1, D2, S2, . . . , particularly of one or more of the reference straps/patterns D1, D2, . . . located at regions not affected by deflections of the sensing sheet, can be used to determine the temperature surrounding the sensor device.

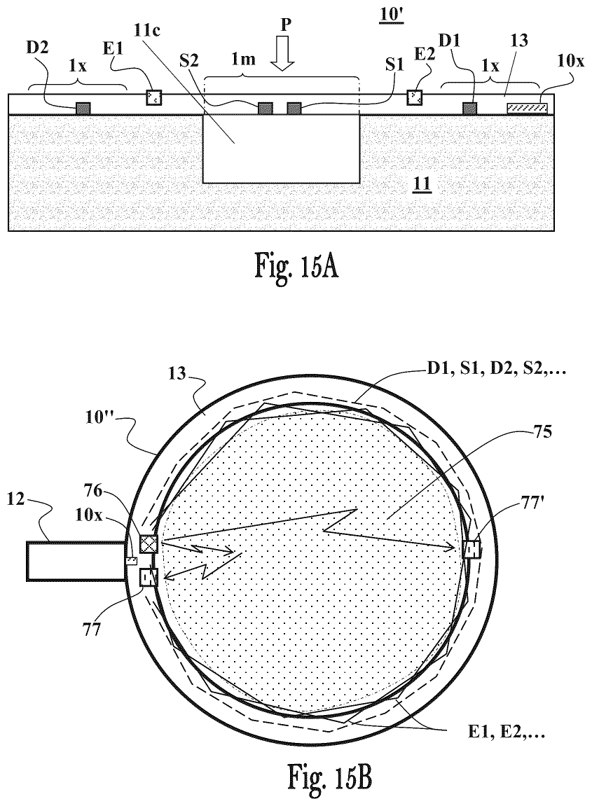

In possible embodiments further sensor elements are added for temperature, and/or electrical resistance, and/or optical transmittance/reflectance, measurements, As exemplified in FIG. 15A. The surface/tactile sensor 10′ shown in FIG. 15A is similar to the surface/tactile sensor of FIG. 1B, but further comprises one or more electrodes E1, E2, . . . (only two electrodes are exemplified in FIG. 15A) configured to measure electrical conductivity of a medium (75 in FIG. 15B e.g., skin/tissue, liquid substance) contacting the surface/tactile sensor 10′. The electrodes E1,E2 can be configured as elongated gauging straps/patterns similar to the gauging straps/patterns 10 D1, S1, D2, S2, . . . , having at least some surface area exposed, or protruding out of the flexible/deformable sheet 13 for establishing physical contact with the external medium (75).

Accordingly, the different sensor embodiments disclosed herein can be configured to at least partially enclose a medium (e.g., tissue, body liquids or organ, fluid substances, etc.) and measure various properties and/or conditions of the medium thereby at least partially enclosed. For example, the different sensor embodiments disclosed herein can be configured to as a wearable article (e.g., wristwear/bracelet, watch) or integrated into wearable articles (e.g., shirts, pants, sleeves, underwear, or suchlike). FIG. 15B demonstrates a wearable sensor device 10″ made of a flexible/deformable sheet 13 comprising various sensing elements configured to at least partially enclose a medium (e.g., body tissue or organ) 75 and measure one or more properties and conditions thereof.

For example, in possible embodiments the wearable sensor device 10″ comprises the one or more gauging straps/patterns D1, S1, D2, S2, . . . , arranged therein according to any of the embodiments disclosed herein. Alternatively, or additionally, in possible embodiments the wearable sensor device 10″ comprises the one or more electrodes E1, E2, . . . shown in FIG. 15A. Alternatively, or additionally, in some embodiments the wearable sensor device 10″ comprises optical sensing arrangements 76,77 and/or 76,77′ configured for measuring light reflectance (or transmittance) of the medium 75. For example, the optical sensing arrangement 76,77 comprising a light emitter 76 and an adjacently located light detector 77 can be used to measure intensity of the light emitted by the light emitter 76 onto/into the medium 75 and reflected therefrom onto the light detector 77. Additionally, or alternatively, the optical sensing arrangement 76,77′ comprising the light emitter 76 and a remote light detector 77′ can be used to measure intensity of the light emitted by the light emitter 76 onto/into the medium 75 and transmitted therethrough onto the light detector 77.

It is noted that the sensing structures/devices disclosed herein can be embedded in other items which can be flexible/elastic, rigid or semirigid e.g., flexible foils, bags. In addition the elongated gauging straps/patterns D1, S1, D2, S2, . . . can be configured with any suitable geometrical shapes (e.g., in square/rectangular wavy patterns, zigzagged patterns, spiral or helical, etc.)

In possible embodiments the elongated gauging straps/patterns D1, S1, D2, S2, . . . are configured to measure changes in electrical and/or electrochemical properties/conditions external thereto e.g., resistance, capacitance and/or inductance, between at least two (e.g., adjacent) elongated gauging straps/patterns of the sensor device, due to changes in the external environment/medium (e.g., humidity, temperature, amount of water, etc.) i.e., in addition to pressure/force changes measurements. These changes can be measured both if the one or more of the elongated gauging straps/patterns D1, S1, D2, S2, . . . are exposed directly for contact with the external environment and/or if the elongated gauging straps/patterns D1, S1, D2, S2, . . . are covered by outer layers (e.g., as exemplified in in FIGS. 10B to 10F).

In some embodiments the outer layers (e.g., as exemplified in in FIGS. 10B to 10F) covering the elongated gauging straps/patterns D1, S1, D2, S2, . . . are configured to facilitate the sensing of changes in external electrical and/or electrochemical properties/conditions. For example, in possible embodiments a certain covering/outer layer can be configured to improve the sensing sensitivity by changing it dialectic properties responsive to the level of the external humidity, or water concentration, or oxygen concentration, or presence of other elements/materials.

In some embodiments the sensing structures/devices include an antenna (and related circuitries) 10x configured for energy harvesting and/or communication of data/signals with external device, as disclosed in International Patent Publication No. WO 2022/149128, of the same applicant hereof, the disclosure of which is incorporated herein by reference. In possible embodiments the antenna element 10x can be patterned on one side of the sensing sheet/substrate layer carrying the sensing/gauging elements, and/or of the other side of the sensing sheet/substrate layer e.g., using the same material used for patterning the electrical conducting lines/layers (41/72).

For example, the antenna/circuitries 10x can be configured to harvest electromagnetic (e.g., RF) energy produced by the control and power unit 51 of FIG. 8A, for energizing the sensor device to acquire measurement data/signals from its gauging straps/patterns D1, S1, D2, S2, . . . , electrodes E1, E2, . . . , optical sensing arrangements 76,77 and/or 76,77′, and/or other sensing elements thereof, and communicate the same to the control and power unit 51. Accordingly, in the disclosed embodiments wherein the sensor structures/devices are shown with both the electrical connection arm 12 and antenna/circuitries 10x, one of these components can be removed, if so required.

It should also be understood that throughout this disclosure, where a process or method is shown or described, the steps of the method may be performed in any order or simultaneously, unless it is clear from the context that one step depends on another being performed first. It is also noted that terms such as first, second, . . . etc. may be used to refer to specific elements disclosed herein without limiting, but rather to distinguish between the disclosed elements. Relative terms such as “lower,” “upper,” “horizontal,” “vertical,” “above,” “below,” “up,” “down,” “top” and “bottom”, as well as derivatives thereof (e.g., “horizontally,” “downwardly,” “upwardly,” etc.), and similar adjectives in relation to orientation of the described elements/components refer to the manner in which the illustrations are positioned on the paper, not as any limitation to the orientations in which these elements/components can be used in actual applications.

For an overview of several example features, process stages, and principles of the invention, the examples illustrated schematically and diagrammatically in the figures are intended for a pressure/force measurement applications. These pressure/force sensing applications are shown as one example implementation that demonstrates a number of features, processes, and principles used to provide tactile sensory capabilities, but they are also useful for other applications and can be made in different variations. Therefore, the above description refers to the shown examples, but with the understanding that the invention recited in the claims below can also be implemented in myriad other ways, once the principles are understood from the descriptions, explanations, and drawings provided herein. All such variations, as well as any other modifications apparent to one of ordinary skill in the art and useful in tactile sensory applications may be suitably employed, and are intended to fall within the scope of this disclosure.

As described hereinabove and shown in the associated figures, the present invention provides sensor device for sensing interactions with external environments and related methods. While particular embodiments of the invention have been described, it will be understood, however, that the invention is not limited thereto, since modifications may be made by those skilled in the art, particularly in light of the foregoing teachings. As will be appreciated by the skilled person, the invention can be carried out in a great variety of ways, employing more than one technique from those described above, all without exceeding the scope of the claims.

1. A surface sensing device comprising:
an elongated interaction layer configured for interaction with environments external to said surface sensing device;
one or more elongated gauging patterns arranged along a length of said elongate interaction layer to substantially traverse a deformable surface area of said surface sensing device; and
at least one integration layer coating at least some portion of said surface sensing device substantially accommodating said deformable surface area and configured to facilitate direct integration of said surface sensing device to an external surface area of an object.

2. (canceled)

3. The surface sensing device according to claim 1 wherein the one or more elongated gauging patterns comprise at least one elongated sensing pattern and at least one elongated reference pattern arranged along a length of said interaction layer substantially parallel to said at least one sensing pattern, and configured to provide a reference for measurement signals/data from said at least elongated one sensing strap/pattern.

4. The surface sensing device according to claim 3 configured to locate the elongated sensing patterns over an elongated cavity of the object defining a deformable sensing region of the device.

5. The surface sensing device according to claim 3 wherein the at least one elongated reference pattern is arranged on a surface area of said elongated interaction layer configured for reduced or non-observable deformations.

6. The surface sensing device according to claim 3 comprising at least two of the elongated sensing patterns and at least two of the elongated reference patterns.

7. The surface sensing device according to claim 3 wherein the at least one elongated sensing pattern and the at least one elongated reference pattern are configured for bridge circuitry measurements, wherein one voltage dividing branch of said bridge circuitry is arranged to present voltage over one of the elongated sensing patterns, and another voltage dividing branch of said bridge circuitry is configured to present voltage over one of the elongated reference patterns.

8.-9. (canceled)

10. The surface sensing device according to claim 3 comprising an electrical connection region at one end of the elongated interaction layer and configured to electrically connect to at least one of the one or more elongated gauging patterns, and one or more of the following: elongated electrically conducting lines arranged along a length of said elongated interaction layer and configured to electrically connect between electrically conducting lines of said electrical connection region and extremities of the elongated gauging patterns at another end of said elongated interaction layer; one or more vias configured to electrically connect between the elongated gauging patterns arranged on one side of the elongated interaction layer and at least one of the electrically conducting lines of the electrical connection region and the elongated electrically conducting lines arranged on another side of said electrical connection region; electrically connecting patterns configured to connect the one or more vias to the elongated gauging patterns, wherein said electrically connecting patterns are at least partially arranged on or under extremities of said elongated gauging patterns.

11.-14. (canceled)

15. The surface sensing device according to claim 7 configured for interfacing to a control unit wirelessly, or over data/signal communication lines, said control unit configured to at least one of a calibration procedure for calibrating the bridge circuitry and communication of measurement data/signals from said bridge circuitry.

16. (canceled)

17. The surface sensing device according to claim 15 comprising one or more tunable resistive elements electrically coupled to at least one voltage dividing branch of the bridge circuitry, and wherein the calibration procedure comprises adjusting electrical resistance of said one or more tunable resistive elements by the control unit until said bridge circuitry is substantially balanced.

18. The surface sensing device according to claim 15 wherein the control unit is configured with a surface area associated with the one or more elongated gauging patterns to distinguish between application of pressure/force over one or more surfaces on said device and application of pressure/force over a discrete point on said device.

19. The surface sensing device according to claim 1 comprising at least one of the following: an adhesive layer connecting the at least one integration layer to the elongated interaction layer; at least one cover layer configured to cover the elongated gauging patterns, communicate external pressure or forces applied thereover to the interaction layer and/or improve sensing sensitivity of said device; an isolation layer configured between the at least one cover layer and the interaction layer; one or electrodes configured to contact a medium and measure electrical conductivity thereof; one or optical sensing arrangement configured to measure light transmittance and/or reflectance of a medium; an antenna and circuitries for one or both energy harvesting and communicating measurement data/signals with an external device.

20.-22. (canceled)

23. The surface sensing device according to claim 1 comprising multiple layers of said surface sensing device arranged one on top of the other.

24.-25. (canceled)

26. The surface sensing device according to claim 1 wherein one or more of the elongated gauging patterns are configured for measuring changes of one or more electrical and/or electrochemical properties/conditions of a medium or environment external to said device.

27. (canceled)

28. The circumferential sensing device comprising at least one surface sensing device according to claim 1 attached at least partially to a curved or flat portion of the object.

29. The circumferential sensing device of claim 28 configured for at least one of exercising and/or diagnosing annular muscles of a treated/exercised subject and comprising at least one of the following: an elongated core, one or more soft/flexible cover layers applied over at least some portion of said elongated core structure, and a plurality of the surface sensing devices embedded in or on said one or more soft/flexible cover layers and along said elongated rigid core; one or more reinforcing elements comprised in, or carried by, the one or more soft/flexible cover layers, each reinforcing element configured to surround and support at least some portion of one of the surface sensing devices; an actuatable portion having the at least one surface sensing device annularly arranged over a cylindrical portion thereof, and a ball portion movable coupled to said actuatable portion.

30.-32. (canceled)

33. The casting apparatus for applying a cover layer over the circumferential sensing device of claim 28, said casting apparatus comprises: connectable front and rear portions having cavities configured to hold components of said surface sensing device substantially centered thereinside; and an injection port provide in at least one of said front and rear portions and configured for injection of a curable casting material to said cavities.

34.-36. (canceled)

37. A method of preparing a sensing device, the method comprising applying at least one integration layer over at least some portion of an elongated interaction layer for coating at least some portion thereof substantially accommodating a deformable surface area of said elongated interaction layer, said elongated interaction layer configured for interaction with environments external to said sensing device and said at least one integration layer configured to facilitate direct integration of said sensing device to an external surface area of an object, arranging one or more elongated gauging patterns along a length of said elongated interaction layer to substantially traverse its deformable surface area, and at least one of the following: arranging of the one or more elongated gauging patterns to form at least one elongated sensing pattern and at least one elongated reference pattern along a length of the interaction layer substantially parallel to said at least one sensing pattern; forming the elongated sensing patterns over a region of the elongated interaction layer configured to cover an elongated cavity of an object defining a deformable sensing region of the device; arranging the at least one elongated reference pattern on a surface area of the elongated interaction layer configured for reduced or non-observable deformations; arranging at least two of the elongated sensing patterns, and at least two of the elongated reference patterns, on the elongated interaction layer.

38.-41. (canceled)

42. The method according to claim 37 comprising configuring the at least one elongated sensing pattern and the at least one elongated reference pattern for bridge circuitry measurements such that one voltage dividing branch of said bridge circuitry is arranged to present voltage over one of the elongated sensing patterns, and another voltage dividing branch of said bridge circuitry is configured to present voltage over one of the elongated reference patterns.

43.-44. (canceled)

45. The method according to claim 37 comprising forming an electrical connection region at one end of the elongated interaction layer, and configuring said electrical connection region to electrically connect to at least one of the one or more elongated gauging patterns, and at least one of the following: arranging elongated electrically conducting lines along a length of the elongated interaction layer and electrically connecting between electrically conducting lines of the electrical connection region and extremities of the elongated gauging patterns at another end of said elongated interaction layer; forming one or more vias in the elongated interaction layer and electrically connecting through said one or more vias between the elongated gauging patterns arranged on one side of said elongated interaction layer and at least one of the electrically conducting lines of the electrical connection region and the elongated electrically conducting lines arranged on another side of said electrical connection region; forming electrically connecting patterns on the elongated interaction layer and at least partially on, or under, extremities of the elongated gauging patterns for electrically connecting the one or more vias to said elongated gauging patterns; connecting the at least one integration layer to the elongated interaction layer by an adhesive layer; applying at least one cover layer over the elongated gauging patterns and configuring at least one cover layer to communicate external pressure or forces applied thereover to the interaction layer; applying an isolation layer between the at least one cover layer and the interaction layer.

46.-52. (canceled)

53. The method of measuring one or more properties or conditions external to or acting on an object or medium, the method comprising attaching over a flat or rounded surface area of the object or medium one or more surface sensing devices according to claim 1, and acquiring measurement data from the elongated gauging patterns and/or electrodes and/or optical sensing arrangements of said one or more surface sensing devices responsive to pressure or force applied thereover.

54. (canceled)

55. The method of claim 53 comprising one or more of the following: interfacing a control unit to at least one of the surface sensing devices and using said control unit for calibrating the at least one surface sensing device and communicating measurement data/signals therefrom; configuring the control unit with a surface area associated with the one or more elongated gauging patterns to distinguish between application of pressure/force over one or more surfaces on said device and application of pressure/force over a discrete point on said device; interfacing a respective connectivity module to at least some of the surface sensing devices and communicating the measurement data/signals to the control unit wirelessly or over data/signal communication lines by said respective connectivity module; configuring the control unit to adjust one or more tunable resistive elements electrically coupled to at least one voltage dividing branch of a bridge circuitry implemented in at least one of the surface sensing devices until said bridge circuitry is substantially balanced; placing the object in a cavity or passage in a body of a treated subject, and using the measurement data for diagnosing said cavity or passage and/or for exercising annular muscles associated with said cavity or passage; centering the object with the surface sensing devices thereby carried in one or more cavities defined inside a casting device and injecting a curable casting material to said cavities via an injection port provide of said casting device; measuring one or more properties or conditions of the medium at least partially enclosed by the one or more surface sensing devices.

56.-61. (canceled)
